(19) United States
(12) Patent Application Publication
(10) Pub. No.: US 2014/0039484 A1
(43) Pub. Date: Feb. 6, 2014

(54) Methods for creating a channel through an occlusion within a body vessel

(71) Applicant: Baylis Medical Company Inc., Mississauga (CA)

(72) Inventor: Ramsey Leung, Toronto (CA)

(73) Assignee: Baylis Medical Company Inc., Mississauga (CA)

(21) Appl. No.: 14/049,449

(22) Filed: Oct. 9, 2013

Related U.S. Application Data
(63) Continuation-in-part of application No. 12/923,292, filed on Sep. 14, 2010.
Application No. 12/923,292 is a continuation-in-part of application No. 11/520,754, filed on Sep. 14, 2006, now Pat. No. 7,828,796.
Application No. 11/520,754 is a continuation-in-part of application No. 11/265,304, filed on Nov. 3, 2005, now Pat. No. 7,947,040.
Application No. 11/265,304 is a continuation-in-part of application No. 10/666,301, filed on Sep. 19, 2003, now Pat. No. 7,048,733.
Application No. 12/923,292 is a continuation-in-part of application No. 11/627,406, filed on Jan. 26, 2007, now Pat. No. 8,092,450.
Application No. 11/627,406 is a continuation-in-part of application No. 13/410,868, filed on Mar. 2, 2012.
(60) Provisional application No. 60/596,297, filed on Sep. 14, 2005.
(60) Provisional application No. 60/743,181, filed on Jan. 27, 2006.
(60) Provisional application No. 60/827,458, filed on Sep. 29, 2006.
(60) Provisional application No. 61/448,578, filed on Mar. 2, 2011.

(51) Int. Cl. A61B 18/18

(52) U.S. Cl. CPC A61B 18/18
USPC 606/33

(57) ABSTRACT

A method is disclosed for creating a channel through an occlusion located in a vein that drains the central nervous system of a patient. The method utilizes a channel creating apparatus, including an energy delivery component operatively coupled to a distal end portion of the channel creating apparatus, to create a channel through the occlusion by delivering energy using the energy delivery component, in order to substantially increase blood flow through said vein to treat chronic cerebrospinal venous insufficiency (CCSVI).

### REFERENCES TO PARENT AND CO-PENDING APPLICATIONS

This application is a Continuation-in-part of U.S. patent application Ser. No. 12/926,292, filed on Nov. 8, 2010 which is a Continuation-in-part of U.S. patent application Ser. No. 11/520,754, filed on Sep. 14, 2006, now U.S. Pat. No. 7,828,796, which is a continuation-in-part of U.S. patent application Ser. No. 11/265,304, filed Nov. 3, 2005, now U.S. Pat. No. 7,947,040 which is a continuation-in-part of U.S. application Ser. No. 10/666,301, filed Sep. 19, 2003, now U.S. Pat. No. 7,048,733, and which is a continuation-in-part of U.S. application Ser. No. 10/760,479, filed Jan. 21, 2004, and which is a continuation-in-part of U.S. application Ser. No. 10/666,288, filed Sep. 19, 2003, which is a continuation-in-part of U.S. application Ser. No. 10/347,366, filed Jan. 21, 2003, now U.S. Pat. No. 7,112,197. Ser. No. 11/520,754 also claims the benefit of U.S. provisional patent application Ser. No. 60/596,297, filed Sep. 14, 2005.

U.S. patent application Ser. No. 12/926,292 is also a Continuation-in-part of U.S. patent application Ser. No. 11/627,406, filed on Jan. 26, 2007, now U.S. Pat. No. 8,092,450, which claims the benefit of U.S. Provisional Patent Application Ser. No. 60/743,181, filed on Jan. 27, 2006 and U.S. Provisional Patent Application Ser. No. 60/827,458, filed on Sep. 29, 2006.

This application is also a Continuation-in-part of U.S. patent application Ser. No. 13/410,868 filed on Mar. 2, 2012, which claims the benefit of U.S. Provisional Patent Application Ser. No. 61/448,578, filed on Mar. 2, 2011.

All of these patents, patent applications and provisional patent applications are hereby incorporated by reference in their entirety.

### TECHNICAL FIELD

The disclosure relates to a method of creating a channel through tissue in a region of a patient's body. More specifically, the disclosure relates to a method of creating a channel through an occlusion located in a body vessel of a patient by delivering energy into the occlusion.

### SUMMARY OF THE DISCLOSURE

In one broad aspect, embodiments of the present invention comprise a method for creating a channel through an occlusion located in a vein that drains the central nervous system of a patient, said method using a channel creating apparatus including an energy delivery component operatively coupled to a distal end portion of said channel creating apparatus, said method comprising: creating a channel through said occlusion by delivering energy into said occlusion using said energy delivery component to substantially increase blood flow through said vein to treat the chronic cerebrospinal venous insufficiency.

As a feature of this broad aspect, the energy comprises radiofrequency electrical energy. As an example of this feature the energy comprises pulsed radiofrequency energy. As another example of this feature, radiofrequency energy is delivered for between about 0.5 seconds to about 2.5 seconds.

As another feature of this broad aspect said vein is selected from the group consisting of: an internal jugular vein, an azygous vein, a subclavian vein and a brachiocephalic vein.

As additional feature of this broad aspect, the method further comprises, prior to creating the channel, a step of orienting the apparatus distal end portion within the vein to follow a trajectory of the vein to steer the apparatus distal end portion substantially adjacent said occlusion.

As an example of this feature, said apparatus distal end portion is angled to facilitate the step of orienting the apparatus distal end portion. In one specific example said apparatus distal end portion has an angle of between about 20 degrees to about 40 degrees with respect to a longitudinal axis of the apparatus.

As an additional feature of this broad aspect, the method further comprising a step of advancing a dilation catheter over the apparatus within the channel created by the apparatus in order to dilate the channel.

As an example of this feature, the method comprises the steps of: withdrawing the dilation catheter; advancing a balloon catheter over the apparatus within the channel created by the apparatus; and inflating the balloon to further dilate the channel. In one embodiment, the step of inflating the balloon is repeated at various locations within the channel in order to further dilate the channel. In an alternative embodiment, the vein includes multiple occlusions and the step of inflating the balloon is repeated at each of said occlusions. In still another embodiment, the method further comprises a step of deploying a stent within said channel.

As another feature of this broad aspect, the method further comprises a step of attempting to cross the occlusion mechanically prior to the step of creating a channel by delivering energy. As an example of this feature, the step of attempting to cross the occlusion mechanically is performed using a mechanical guide wire. As another example of this feature, the step of attempting to cross the occlusion mechanically is performed using the channel creating apparatus.

As another feature of this broad aspect, said channel is created into or through said occlusion located within a region of the vein having a stent installed therein. As an example of this feature, the method further comprises a step of: detecting an error and stopping the delivery of energy into said occlusion using the energy delivery component if the energy delivery component is positioned in proximity to the stent or substantially contacts said stent.

In another broad aspect, embodiments of the present invention include: a method for creating a channel through a site of an occlusion located in a vein that drains the central nervous system of a patient, said occlusion at least partially restricting the flow of blood within the vein contributing to chronic cerebrospinal venous insufficiency said method using a channel creating apparatus comprising a radiofrequency guidewire, the channel creating apparatus including an energy delivery component operatively coupled to a distal end portion of said channel creating apparatus, said method comprising the steps of: advancing a mechanical guide-wire within the vein substantially adjacent the occlusion; applying contrast at the site of the occlusion to visualize the extent of the occlusion; advancing the radiofrequency guidewire to the site of the occlusion such that the apparatus distal end portion is positioned adjacent the occlusion; attempting to advance through the occlusion mechanically substantially without the delivery of radiofrequency energy using the radiofrequency guidewire; and, upon being unable to advance the radiofrequency guidewire mechanically, creating a channel through said occlusion by delivering energy into said occlusion using said energy delivery component to substantially increase blood flow through said vein to treat chronic cerebrospinal venous insufficiency.

As a feature of this broad aspect, the steps of attempting to advance through the occlusion mechanically and creating a channel through the occlusion are repeated in order to cross the occlusion to treat chronic cerebrospinal venous insufficiency while reducing the risk that energy delivery may injure tissues that should remain intact.

### BRIEF DESCRIPTION OF THE DRAWINGS

In order that the invention may be readily understood, embodiments of the invention are illustrated by way of examples in the accompanying drawings, in which:

FIGS. 1A-1B are an illustration of an electrosurgical device in accordance with an embodiment of the present invention;

FIG. 2 is an illustration of an electrosurgical device in accordance with an alternate embodiment of the present invention;

FIG. 3 is an illustration of an electrosurgical device in accordance with an alternate embodiment of the present invention;

FIG. 4 is an illustration of an electrosurgical device in accordance with an alternate embodiment of the present invention;

FIG. 5, in a flow chart, illustrates a method for creating a channel in accordance with an embodiment of the present invention;

FIGS. 6A-6C illustrate a channel created in an occlusion of a body vessel using the method illustrated in FIG. 5;

FIGS. 7A-7C show an elongate member of an electrosurgical device in accordance with various embodiments of the present invention;

FIG. 8 illustrates a partial side cross-sectional view of an alternate embodiment of an electrosurgical device; and

FIGS. 9A-9E show the electrode in accordance with various embodiments of the present invention.

### DETAILED DESCRIPTION

According to the embodiments described herein below, an occlusion may refer to a condition that at least partially occludes the blood vessel and restricts blood flow within the vessel. An occlusion may refer to, for example, a narrowing of a blood vessel due to a partial occlusion or a blockage, a chronic total occlusion, stenosis or avascular malformation. Thus, occlusion may refer to any such obstruction that at least partially restricts blood flow through the vessel.

Embodiments of the present invention comprise a method for creating a channel through an occlusion located in a vein that drains the central nervous system of a patient, said method using a channel creating apparatus including an energy delivery component operatively coupled to a distal end portion of said channel creating apparatus, said method comprising: creating a channel through said occlusion by delivering energy into said occlusion using said energy delivery component to substantially increase blood flow through said vein to treat chronic cerebrospinal venous insufficiency (CCSVI).

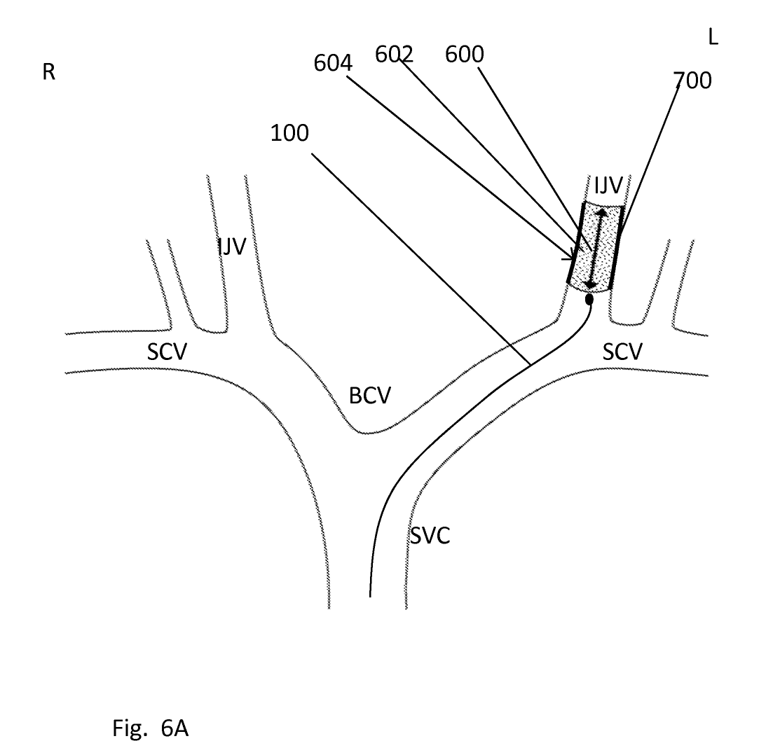

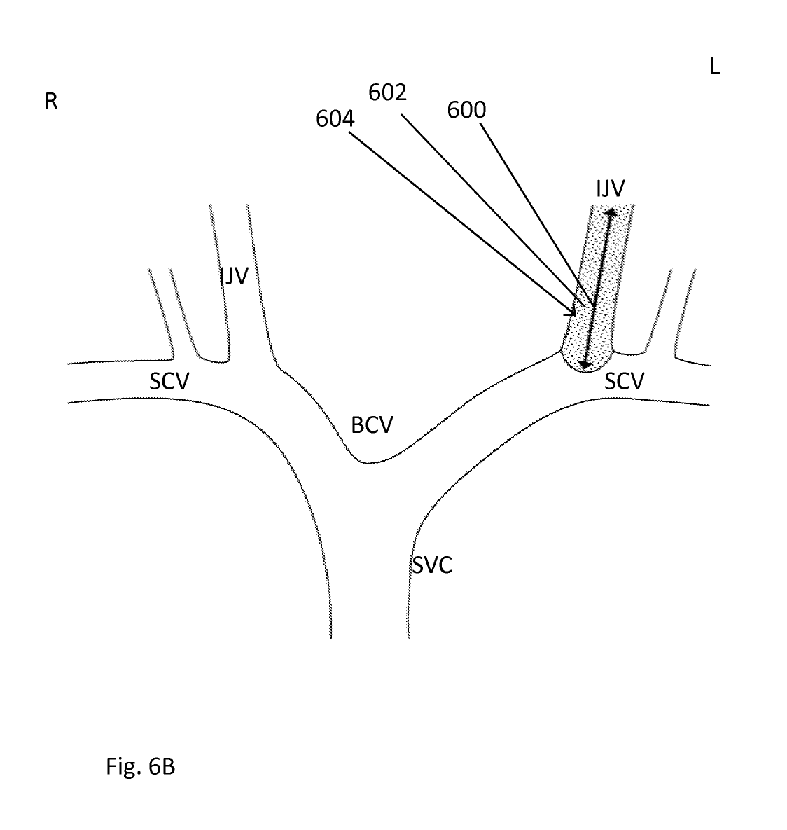

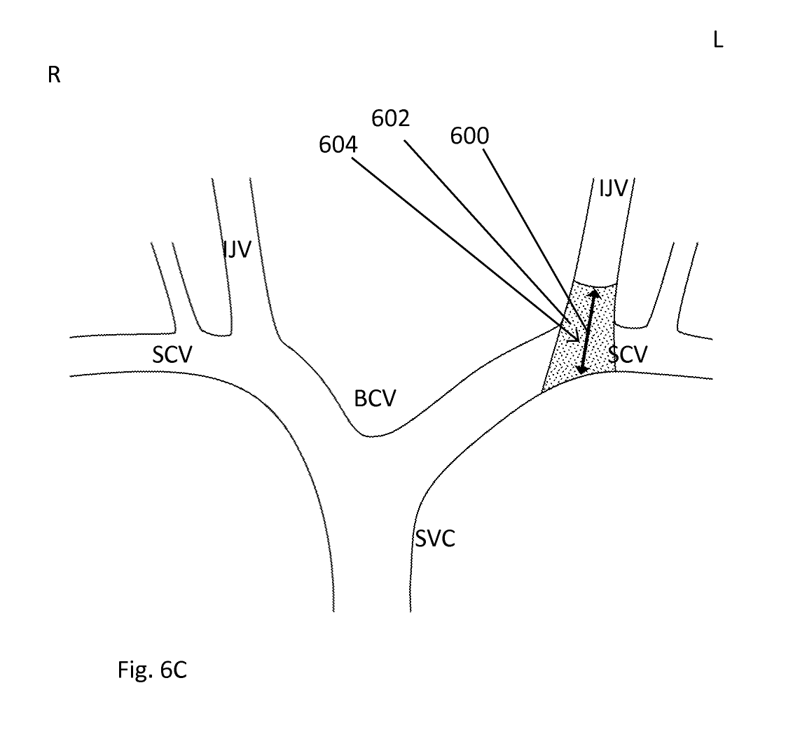

In a particular example of such an embodiment, the azygos and/or the internal jugular veins (IJV) may be occluded, for example, the veins may be stenotic (abnormally narrowed). In other examples, the occlusion may alternatively or additionally involve the subclavian veins (SCV) and/or brachiocephalic/innominate veins (BCV). Additionally, truncular venous malformations, including azygous stenosis, defective jugular valves and jugular vein aneurysms, and problems within the superior vena cava (SVC) may also to contribute to CCSVI. Truncular venous malformation lesions may cause stenosis along the internal jugular (IJV), innominate, superior vena cava (SVC) and azygos vein system, contributing to CCSVI. In some embodiments, inverted valves, scarring of valves or stenosis around the valves and membranes all may cause valves to malfunction and may contribute to CCSVI. The internal jugular veins (IJV), subclavian veins (SCV), brachiocephalic veins (BCV) and the superior vena cava (SVC) are all illustrated in FIGS. 6A-6C.

Such medical conditions may be treated by use of a medical device to traverse or cross the occlusion for example to allow a balloon to be advanced along it and deployed thereafter in order to treat the occlusion. In one such example, a mechanical guide wire may be used that may be inserted through a blood vessel such as a vein within the patient and advanced to the site of the occlusion such as within the IJV. The mechanical guide wire may then be advanced substantially longitudinally through the occlusion. Once a passage has been created through the occlusion, a balloon may be then advanced over the guide wire to the site of the occlusion to dilate the channel through the occlusion to allow increased blood flow along the vessel at the site of the occlusion.

Although energy-based devices have been used to treat occlusions in other body vessels, the risk of perforating or puncturing the sensitive anatomical structures implicated in CCSVI is significantly higher than, for example, the peripheral limbs of a patient's body. Therefore, traversal of such occlusions is primarily performed using mechanical guide wires.

Oftentimes, the occlusion that is being encountered may be especially tough or calcified and the practitioner may find it difficult to cross the occlusion. Practitioners have been inclined to use mechanical guide wires as a standard of care for treating occlusions such as within the IJV to ensure patient safety. However, the use of such mechanical guide wires to traverse through a difficult or tough occlusion may require the practitioner to exert a large amount of force using the mechanical guide wire to try and maneuver the guide wire through the occluded vessel. This may result in force being applied in an uncontrolled manner which may be detrimental to the vessel wall in terms of increasing the risk of inadvertently puncturing the vessel wall.

The present inventors have discovered a method of using a device that is capable of delivering energy in order to treat an occlusion within vessels that drain the central nervous system in a safe and effective manner. In accordance with this approach, the present inventors have reduced to practice embodiments of devices that employ energy, such as, for example, electrical energy in the radiofrequency (RF) range, for treating occlusions that may be causative of CCSVI while reducing/minimizing risk to the patient. Such embodiments are particularly useful and advantageous, for example, when the occlusion is particularly tough or fibrotic and cannot easily/readily be penetrated using a mechanical guide wire and/or when the occlusion has been previously treated by stenting but has now re-stenosed and cannot easily/readily be re-canalized using mechanical intervention.

The use of the device of the present invention is more controlled in comparison to utilizing mechanical force and traditional/mechanical guide wires to cross an occlusion within the sensitive vasculature critical for adequate drainage of blood flow from the brain. It provides for controlled delivery of both RF and mechanical force in order to traverse the occlusion within vessels such as the IJV. In one specific example, the embodiments of the present invention provide for delivery of RF in a pulsed mode or over short durations. Thus, it may allow for both a lower amount of force to be applied as well as the RF energy to be applied in a more controlled and targeted manner to direct it at the occlusion within the vessel. This may allow, for example, for minimizing the narrowing within the blood vessel by creating a channel through the occlusion to increase blood flow. Furthermore, as the device creates a channel through the occlusion and is advanced therethrough, it may allow a secondary device such as, for example, a balloon to be advanced over the device to allow for angioplasty for further dilation of the channel to further increase blood flow through the vessel.

More specifically, embodiments of the present invention avoid purely mechanical means to traverse a tough occlusion to reduce the risk of vessel perforation from exertion of uncontrolled or large amounts of force. Exemplary embodiments of the present invention provide an apparatus such as a device 100 that may be an electrosurgical device, as described herein below, that is operable to deliver radiofrequency (RF) electrical energy from an electrode at the distal tip thereof, once the medical device 100 has been advanced within the vasculature to the site of the occlusion in order to treat the occlusion. The device delivers electrical energy to initiate arcing at the electrode to perforate or vaporize the occlusion in order to create a channel therethrough to increase blood flow through the vessel at the site of the occlusion.

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The description provided herein below for device 100 will be taken by a person skilled in the art to apply to any one the devices 100A-100E shown below.

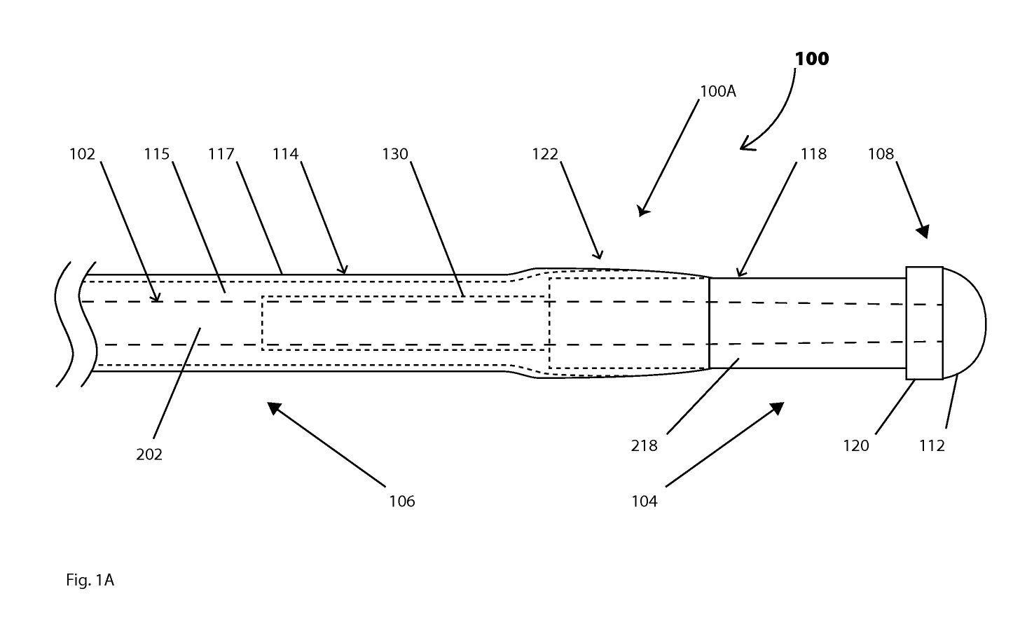

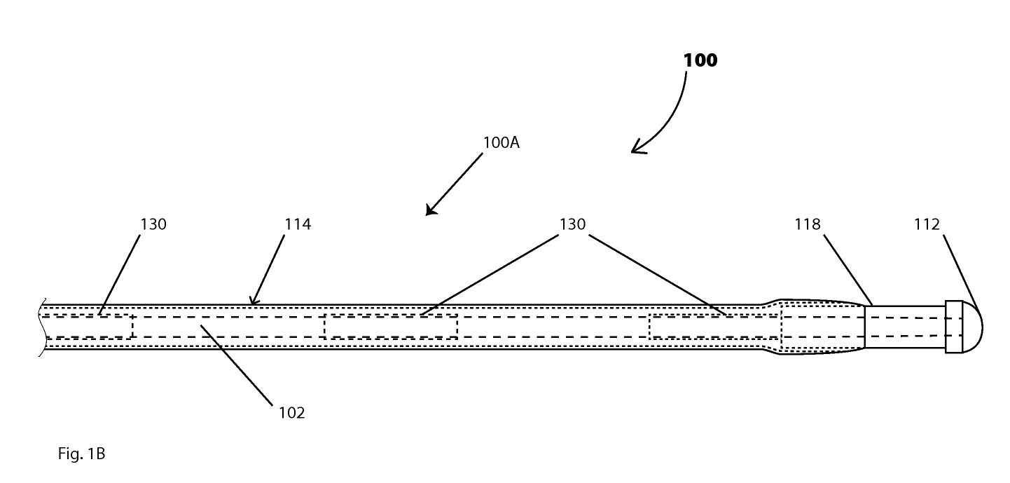

In accordance with an embodiment of the present invention, as shown in FIG. 1A, the device 100 such as an electrosurgical device 100A comprises an inner elongate member 102 which is an electrical conductor. A heat shield 118 is positioned at the distal end of the elongate member. An electrode tip 112 is coupled to the distal end of the elongate member 102 distal to the heat shield 118. In one example, a support structure 120 is positioned distal to the heat shield 118 for supporting the electrode tip 112. The elongate member has an insulation layer 114 disposed along a portion thereof including along a proximal region 106 of the device. The device proximal region is the portion of the device 100 that is proximal to the heat shield 118.

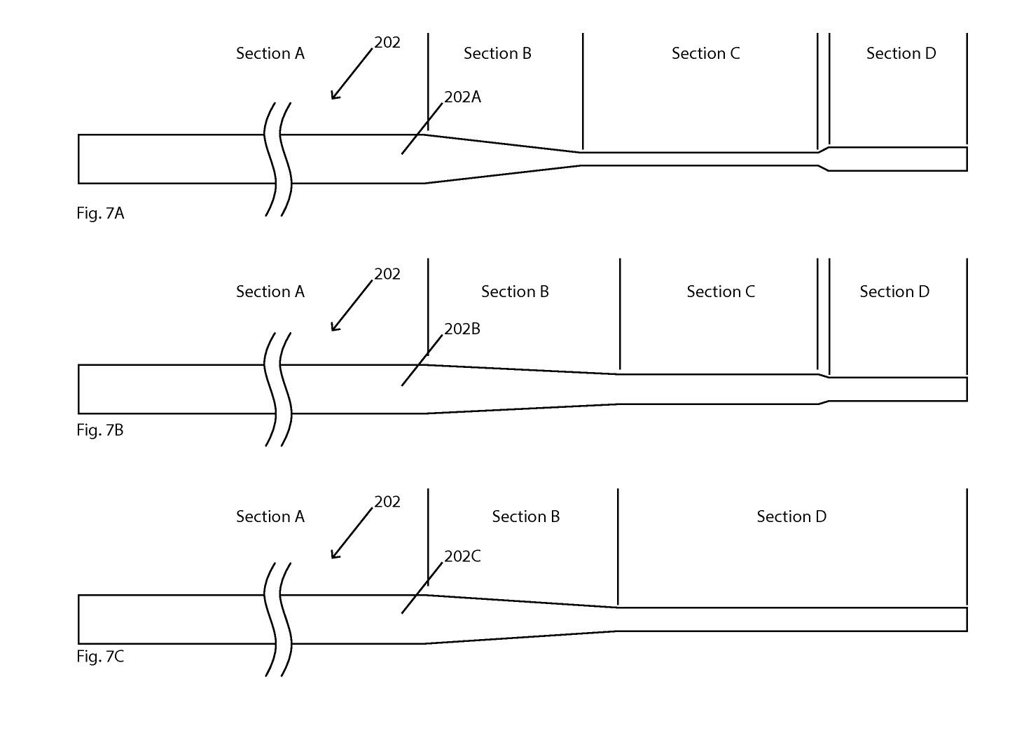

In accordance with an embodiment of the present invention, the electrosurgical device 100 may comprise an inner elongate member 102 that may comprise an electrical conductor. The inner elongate member 102 extends longitudinally and has a proximal end coupled to an energy source and a distal end coupled to an energy delivery component, such as an electrode having an electrode tip 112. In one embodiment, the inner elongate member 102 may comprise a core wire 202. In one example, the core wire 202 may comprise a shape memory alloy, such as a nickel-titanium alloy. One example of a nickel-titanium alloy is Nitinol™. As shown in FIG. 1A, in one instance of this example, the elongate member comprises a Nitinol core wire 202. In another instance of this example, the core wire 202 may comprise stainless steel. Alternatively, any other suitable material may be used for core wire 202. In some embodiments the elongate member 102 may have an outer profile defined by a uniform outer diameter along its length. In other embodiments, the elongate member 102 may have a variable outer diameter along its length. In some examples, the elongate member 102 comprising a core wire 202 has varying outer diameters along its length as shown by core wires 202A, 202B and 202C in FIGS. 7A-7C. This may provide varying degrees of stiffness and flexibility along different sections of the core wire 202. The varying diameters of the core wire 202 and the thickness of the insulation layer 114 along the distal region of the device can help control/modify the stiffness of the device distal region. A smaller outer diameter in a section of the core wire 202 may provide increased flexibility of the wire in that region and may allow the wire to be floppier in that region. In one example, the core wire 202 has sufficient flexibility to allow the device 100A to conform to the vasculature and has sufficient rigidity to allow pushability of the device 100 through tissue. Alternatively, the elongate member 102 may have any other suitable profile. In one example, the elongate member 102 may comprise a solid electrical conductor. In another example, the elongate member 102 may be hollow. In one example, the elongate member 102 may not be hollow to increase rigidity of the device 100.

In some embodiments of the present invention the electrosurgical device 100 may have a predefined curvature. A portion of the electrosurgical device such as distal region 104 can be provided with varying degrees of curvature which may help in navigation of the device 100 through vasculature. In one example, the curvature of the electrosurgical device 100 may facilitate the device 100 in engaging with an occlusion located in peripheral vasculature. In one specific example, the curvature of the electrosurgical device 100 may allow the device 100 to engage an occlusion located at or near a bifurcation in a body vessel. In some embodiments, the curvature of device 100 may be provided by using a shape memory alloy for the elongate member 102 that has been shape set to a preset curvature. In one example, the elongate member 102 may comprise a Nitinol core wire 202 which may be shape set with the specific angle of curvature or shape that is needed. In examples, where a shape memory alloy such as Nitinol core wire 202 is used, the shape memory alloy can be treated to have super-elastic properties so that it will not deform permanently when it is pushed against an occlusion. The core wire 202 can go back to its original shape when it is retracted minimizing the risk of the wire distal end being bent out of shape. In other embodiments, the elongate member 102 may have a bend or angle at a location along its length. In still other embodiments, the elongate member 102 may comprise a core wire 202 that comprises a hollow hypotube such as a metal hypotube that may be laser cut to provide flexibility at the distal tip. In still other embodiments the core wire 202 may comprise a coil disposed onto a distal portion of the core wire 202 in order to increase flexibility in the distal portion. Furthermore, the device 100 may be a steerable device.

In some embodiments, the electrosurgical device 100 may have an insulation layer 114 disposed along a portion of the device 100. In some embodiments the insulation layer 114 may be disposed along most of the length of the device including along a proximal region 106 of the device. In other embodiments, the insulation layer 114 may be disposed substantially only along the proximal region 106 of the device. The insulation layer 114 may help to electrically insulate a portion of the electrosurgical device 100. This may help protect the patient and the user for e.g. the physician from current during use of device 100. In one example, the insulation layer 114 is disposed onto a core wire 202 of device 100A, substantially along the proximal region 106 of device 100A. In one embodiment the insulation layer 114 is disposed on the elongate member 102, after the distal components comprising the thermal or heat shield 118, the electrode tip 112 or additionally support structure 120 have been coupled to or formed onto the elongate member 102 distal end. In other embodiments, the elongate member 102 may be coated with an insulation layer 114 prior to the distal components being coupled to the elongate member distal end (not shown). In other words, the elongate member 102 may be provided as an insulated elongate member 102 having an insulation layer 114. In one such example, the heat shield 118 may be loaded onto a distal end of an insulated core wire 202 that is at least partially insulated. A support structure 120 may be loaded onto the core wire 202 and may be electrically in contact therewith, the support structure 120 being positioned distal to the heat shield 118. Additionally, an electrode tip 112 may be formed integrally with the core wire 202 onto the support structure 120. In still other embodiments, the elongate member 102 may be provided as an insulated core wire 202 which can be coated with an additional insulation layer 114 after distal components have been coupled to the core wire 202 distal end.

A variety of materials may be used for the insulation layer 114, including but not limited to polymer or ceramic. In one instance the insulation layer 114 may comprise a polymer insulation layer which may be provided using a heat shrink process or a melt processing method. Alternatively any other suitable method may be used. In some embodiments, the insulation layer 114 may be provided through a dip coating process. A portion of the electrosurgical device 100 or the elongate member 102 may be dipped in a liquid for e.g. a liquid polymer such as liquid PTFE or a ceramic. In other embodiments, a portion of the device 100 or the elongate member 102 may be spray coated with an insulative material for e.g. a polymer or a ceramic. In still other embodiments, a vapor deposition technique may be used to form the insulation layer 114. In some embodiments, where an insulated elongate member 102 is provided, the elongate member 102 may be dip coated with a polymer or insulated with a thin PTFE layer to form the insulated elongate member 102.

In one embodiment, a polymer combination may be used for the insulation layer 114, as shown in FIG. 1A. As an example, a two layer heat shrink layer may be used comprising an inner polymer layer 115 and an outer polymer layer 117. In another example, the inner polymer layer 115 may comprise a melt-processable polymer that can flow around and into any irregularities on the surface of core wire 202 and the outer polymer layer 117 may comprise a heat recoverable polymer. In a specific instance of this example, the insulation layer 114 comprises a combination of FEP and PTFE polymers, where the inner polymer layer 115 comprises FEP and the outer polymer layer 117 comprise PTFE as shown in FIG. 1A. A process combining re-flow and heat-shrink is used and the dual polymer layer is heated to a temperature of about 660° F., allowing the inner FEP layer to flow around and encapsulate the one or more radiopaque bands 130, which, in one specific example, comprise platinum, disposed on core wire 202. Whereas, the outer PTFE layer recovers to a pre-specified diameter around the FEP and provides a smooth outer finish. In another instance of this example, PEBAX may be used as a melt-processable inner polymer layer. In one embodiment, one or more radiopaque bands 130 may be disposed along the core wire 202. The radiopaque bands 130 may comprise, for example, radiopaque material such as gold, iridium or platinum. In one example, a plurality of radiopaque bands 130, are disposed along the core wire 202 with each of the radiopaque bands comprising platinum, as shown in FIG. 1B. In one embodiment, the insulation layer 114 may be disposed over the radiopaque bands 130. The insulation layer 114 may provide a smooth outer profile for the electrosurgical device 100.

In one embodiment of the present invention, a portion of the electrosurgical device 100 may have a hydrophilic coating disposed thereon. This may help make the device lubricious and may help the device 100 to traverse through vasculature or through tissue, for e.g. an occlusion. The insulation layer 114 may comprise a material that allows a hydrophilic coating to be disposed thereon. In some embodiments the insulation layer 114 may comprise a polymer such as PEBAX, FEP or other non-fluoro polymers onto which a hydrophilic coating can be applied. In some embodiments, as mentioned previously, a combination of polymer layers may be used to form the insulation layer 114, for e.g. an inner polymer layer 115 and an outer polymer layer 117. In other examples, any number or combination of polymer layers may be possible. In such embodiments, the outer polymer layer may comprise a material that can be coated with a hydrophilic coating. Some non-limiting examples include an inner polymer layer 115 of PTFE and an outer polymer layer 117 of either FEP or Pebax, OR an inner polymer layer 115 of FEP and an outer layer 117 of Pebax. In some embodiments, the inner polymer layer 115 may provide electrical insulation and the outer polymer layer 117 may allow the device 100 to be coated with a hydrophilic coating. In some embodiments, the entire device 100 may be coated with a hydrophilic coating. In other embodiments, the distal region 104 of the device 100 may have the hydrophilic coating which may include the heat shield 118. In other words, the distal region 104 may be made lubricious. In a non-limiting example, the hydrophilic coating may comprise Hyaluronic Acid (HA).

In one embodiment, as shown in FIG. 1A, the electrosurgical device 100 defines a distal region 104 having a heat shield 118 (which may alternatively be referred to as the thermal shield or heat sink) disposed at or near the distal end of the core wire 202 substantially distal to the insulation layer 114. The heat sink or heat shield 118 is positioned between the insulation layer 114 and the energy delivery component, such as electrode tip 112 positioned at the distal end of core wire 202. In one embodiment the electrode is formed substantially by the electrode tip 112. In another embodiment, the electrode tip 112 is the electrode and forms the energy delivery component of electrosurgical device 100. Energy may be delivered by electrosurgical device 100 using the energy delivery component. In one example, the active electrode tip 112 is positioned distal to the heat shield 118 at the distal tip 108. The distal tip 108 defines the part of the distal region 104 that is distal to the heat sink or heat shield.

In some embodiments of the present invention, the heat shield 118 is an electrically insulative thermal shield. A junction 122 forms between the insulation layer 114 and the heat shield 118. The heat shield 118 is an electrical and thermal insulator that functions to insulate and thus protect the device proximal region 106 from the heat generated at the electrode tip 112 and functions to prevent arcing between the electrode tip and the device proximal region 106. The device proximal region 106 is the portion of the device that is proximal to the heat shield 118. In some embodiments, the heat shield 118 has a thermal conductivity that allows the heat shield 118 to dissipate heat by effectively conducting heat away from the electrode tip 112. This may prevent the heat shield 118 from cracking. Thus, the heat shield 118 electrically and thermally protects the device proximal region 106 and thus the insulation layer 114 in the device proximal region 106.

In one embodiment, the heat shield has a thermal conductivity k, that is greater than about 1 Watt/mK. In other embodiments, the heat shield has a thermal conductivity k that is greater than about 2 Watts/mK. In some embodiments the heat shield 118 may comprise glass or a ceramic such as alumina, aluminum oxide, zirconia toughened alumina (ZTA) or zirconium oxide. In one example, the heat shield 118 comprises a ceramic heat shield 218 that is made of pure alumina or sapphire crystal comprising a single/mono crystal aluminum oxide, as shown in FIG. 1A. The ceramic heat shield 218 may additionally have properties as described herein for heat shield 118. In other embodiments, other ceramics such as Silicon Nitride or Silicon carbide may be used. In still other embodiments, any other suitable ceramic may be used for the ceramic heat shield 218. In still other embodiments, the heat shield 118 may comprise any other suitable insulative material that has sufficient thermal conductivity to conduct heat away from the electrode tip 112. Additionally, the heat shield 118 and has sufficient mechanical strength which may allow it to be machined into the desired shape such as a tubular cylindrical shape.

In one specific example, the ceramic heat shield 218 can withstand the changes in temperature initiated at the electrode tip 112. In one such example, the electrode tip 112 is a mono-polar active electrode. When arcing is initiated at the mono-polar active electrode, high temperatures are created at the electrode tip 112 and within the region of tissue surrounding the electrode tip 112. The ceramic heat shield 218 is thermally insulative and withstands high temperatures while maintaining good dielectric properties. Thus, the ceramic heat shield 218 additionally functions as an electric insulator. In some examples, a thermally insulative ceramic heat shield 218 is employed that has some thermal conductivity and transfers heat well. Thus, the ceramic heat shield 218 allows heat to be dissipated away from the active electrode tip 112 to help minimize transmission of heat to the device proximal region 106 having the insulation layer 114. This minimizes thermal damage to the insulation layer 114 from the arcing generated at the electrode tip 112. Thus, a proximal segment of the electrosurgical device 100 is effectively shielded from arcing at the electrode tip 112 as well as from the heat generated at the electrode tip 112. In other words, the ceramic heat shield 218 prevents degradation of the insulation layer 114 due to its proximity to arcing.

Thus, in general, the heat shield 118 acts as a protective barrier between active electrode tip 112 and the insulation layer 114 by electrically insulating the electrode tip 112 from arcing at the electrode tip 112 and by providing thermal protection for the insulation layer 114 by substantially thermally insulating the device proximal region 106 from the electrode tip 112. Furthermore, the heat shield has a thermal conductivity that allows it to conducting heat away from the electrode tip 112, thus providing additional thermal protection for the device proximal region 106 from the heat generated at the electrode tip 112. In other words, a single heat shield 118 functions as a barrier between the device proximal region 106 and the electrode tip 112 and provides provide both the benefit of preventing arcing between the device proximal region 106 and the electrode tip 112 AND protects the device proximal region from the heat produced by the delivery of energy through the electrode tip 112. In one embodiment, the heat shield 118 functions to electrically and thermally isolate the device proximal region 106 including the insulation layer 114 from the device distal tip 108 including the electrode tip 112.

In one specific example as illustrated in FIG. 1A, the heat shield 118 is a ceramic comprising a tubular single crystal aluminum oxide (sapphire) cylinder forming the ceramic heat shield 218 that is a thermal insulator having adequate thermal conductivity and mechanical strength. The sapphire ceramic heat shield 218 can support voltages necessary or used for generating arcing and can withstand higher temperatures resulting from arcing at the distal tip 108. The sapphire ceramic heat shield 218 insulates the device proximal region 106 and additionally has a thermal conductivity that allows it to conduct heat away from the electrode tip 112. Thus the sapphire ceramic heat shield 218 helps maintain the integrity of device 100A by ensuring that the insulation layer 114 remains intact by minimizing the risk of degradation from intense heat. In other words, the sapphire ceramic heat shield 218 protects the proximal region 106 of the device 100A having the insulation layer 114 from the heat generated at electrode tip 112. In one example, the single crystal Aluminum Oxide ceramic (sapphire) heat shield 218 also provides mechanical strength and helps impart rigidity to the distal region 104 of the electrosurgical device. This may help reduce the risk of the heat shield 218 from cracking when the device 100A is pushed or manipulated during the manufacturing process. Thus the ceramic heat shield 218 may both provide thermal conductivity and mechanical strength and/or rigidity. In one specific instance of this example, the ceramic heat shield 218 is a single crystal Aluminum oxide heat shield that is a tubular cylinder having a longitudinal length of about 2.54 mm. The tubular cylindrical ceramic has an inner diameter of about 0.292 mm and an outer diameter of about 0.660 mm.

In some embodiments, the heat shield 118 comprises material that can be viewed under an imaging modality. In one example, the ceramic heat shield 218 is radiopaque. Additionally, as shown in FIG. 1B, the placement of the radiopaque bands 130 onto to the inner core wire 202 may enhance visualization and may also help increase rigidity of the device 100. In one embodiment, the combination of the radiopaque bands 130, the core wire 202 and the ceramic heat shield 218, provides sufficient rigidity to enhance pushability of device 100 through tissue, for e.g. an occlusion. In other words the device 100, such as device 100A may additionally have rigidity that is imparted by core wire 202, the radiopaque bands 130 and the heat shield 118. In one embodiment, as shown in FIG. 1A, a radiopaque band 130 is positioned proximal to the heat shield 118. In one example, the radiopaque band 130 is secured to the core wire 202 to retain and/or support the heat shield 118 in position. In one specific instance of this example, the radiopaque band 130 is spot welded to the core wire 202. In another example, the heat shield 118 is retained and/or supported in place by the core wire 202. The core wire 202 has wider section adjacent and proximal to the distal section as shown in FIG. 7B. The heat shield 118 is loaded on the distal section and retained by this wider section of the core wire 202. In one embodiment, as shown in FIG. 1B multiple radiopaque bands 130 are positioned on the core wire 202. These provide reference markings which when viewed under imaging provide guidance to the physician for positioning the device 100A within a patient's body and/or advancement of the device 100A during use. The radiopaque bands 130 may comprise materials such as platinum, iridium, gold, silver, tantalum and tungsten or their alloys, or radiopaque polymer compounds. In one example, as mentioned above, platinum is used for the radiopaque bands 130.

As outlined previously, a junction 122 forms between the insulation layer 114 and the heat shield 118. In some examples of this, a seamless transition is provided at the junction 122 between the insulation layer 114 and the ceramic heat shield 218. In one embodiment, the insulation layer 114 extends over the ceramic heat-shield 218 at the junction 122 as shown in FIG. 1A. Therefore, there is an overlap of insulation layer 114 over the ceramic heat shield 218 forming a sealed junction. In one specific example, the insulation layer overlaps the proximal portion of the ceramic heat shield 218 by about 1 mm. The overlap of the insulation layer 114 at junction 122 and the ceramic heat shield 218 help to limit the arcing to the electrode tip 112 at the distal tip 108. This may help minimize arcing observed behind the heat shield near the junction 122 and may help minimize degradation of the insulation layer 114 from the heat generated at the electrode tip 112 from the delivery of electrical energy through the electrode tip 112. As mentioned above, the heat shield 118 is an electrical insulator and contributes to the prevention of arcing between the electrode tip 112 and the device proximal region 106, the proximal region being proximal to the heat shield 118. Additionally the heat shield 118 is thermally insulative and insulates the device proximal region 106. Furthermore, the heat shield 118 has thermal conductivity that allows it to conduct heat away from the electrode tip 112 to thermally protect the device proximal region comprising the insulation layer 114. Thus, the heat shield 118 eletrically and thermally protects the device proximal region 106 from arcing and heat generation at the electrode tip 112 during use.

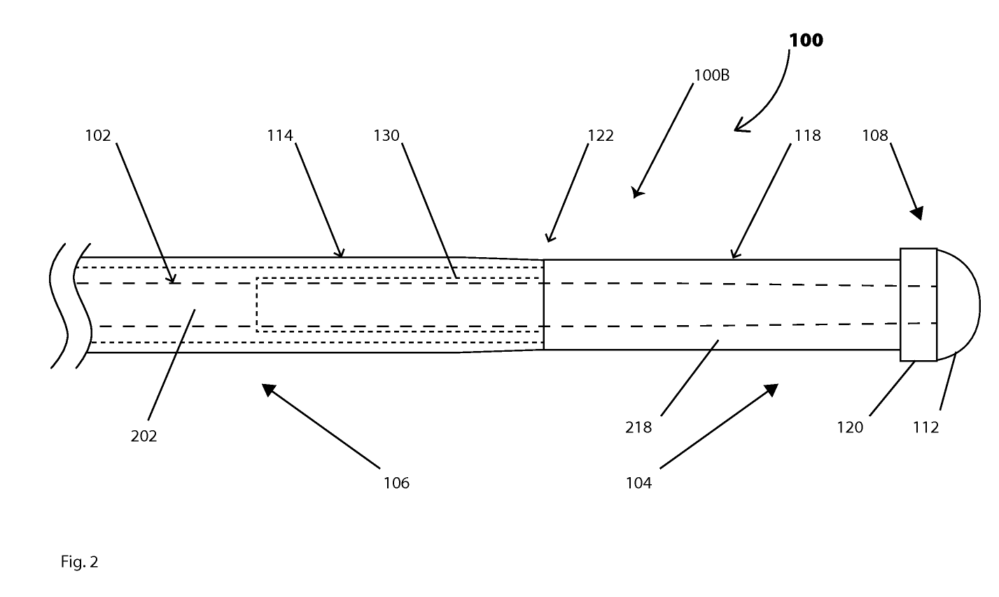

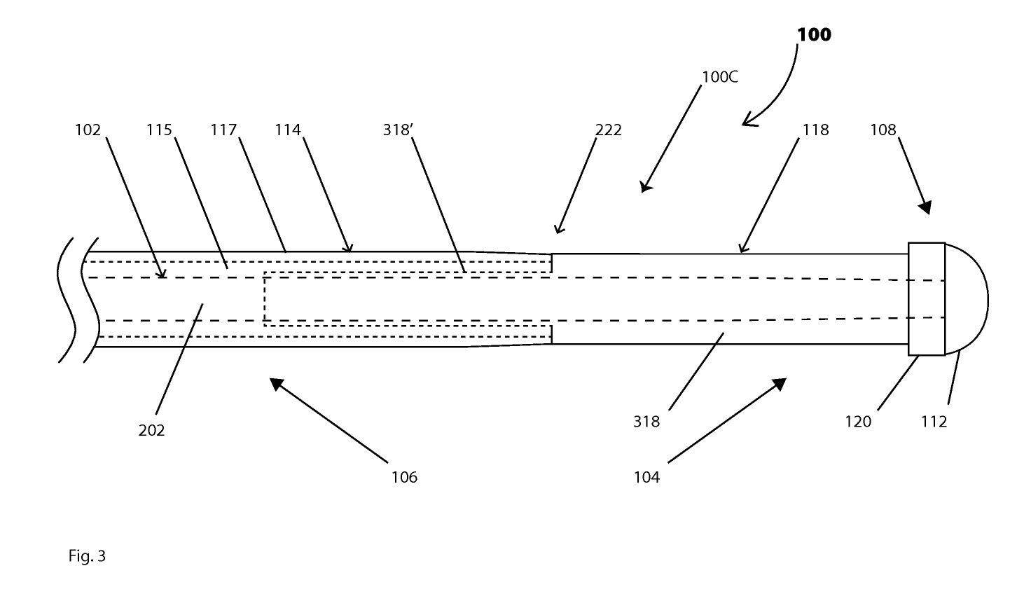

In another embodiment, the insulation layer 114 and the heat shield 118 such as ceramic heat shield 218 may be flush against one another (or in other words abut one another) to form a junction there-between as shown by device 100B in FIG. 2. In still another embodiment, a step-down heat shield 318 may be used as shown by device 100C in FIG. 3. The heat shield 318 may be a ceramic heat shield having properties similar to those mentioned previously for ceramic heat shield 218. Additionally the description provided herein for heat shield 118 will be taken by POSITA to also apply to heat shield 318. The heat shield 318, for e.g. a tubular ceramic heat shield, is wider at its distal portion than at its proximal portion. This allows the insulation layer 114 to form around the proximal portion 318′ of the heat shield 318. The insulation layer 114 is formed flush against and surrounds the proximal portion 318′ of the heat shield 318. This allows for a smooth transition between the heat shield 318 and the insulation layer 114, and may also allow for a secure junction 222 to be formed between the two materials. This helps ensure that the core wire 202 is not exposed at the junction 222. This also helps minimize any shoulder effect which may otherwise be created between the insulation layer 114 and the heat shield 318. Thus, the risk of a discontinuity forming due to exposure of core wire 202 at the junction 222 is minimized. This allows arcing to be limited the electrode tip 112. Furthermore, the smoother outer profile at the junction 222 may prevent tissue from getting caught and snagging at the junction 222 which may allow device 100C to traverse through the occlusion relatively easily.

In some embodiments of the present invention, energy is supplied from the energy source through the elongate member 102 to the energy delivery component comprising electrode tip 112. The electrode tip 112 is coupled to the elongate member distal end which receives energy from the energy source. The electrode tip 112 is configured and sized such that a sufficiently high current density is provided at the electrode tip 112 to generate arcing in a region of tissue when the electrode tip 112 is positioned proximate the region of tissue. This allows a channel to be created through at least a portion of the region of tissue. “proximate” is defined as the electrode tip 112 being positioned either substantially adjacent to the tissue OR being slightly spaced apart from the tissue such that a gap exists between the tissue and the electrode tip 112. In one example, delivery of energy through electrode tip 112 of device 100 allows an occlusion such as a harder occlusion to be traversed. In other words the electrode tip 112 is shaped to provide a sufficient current density to initiate arcing at the electrode tip 112 during the delivery of energy which may allow the electrosurgical device 100 to create a channel through at least a portion of the occlusion or region of tissue. The occlusion may comprise an occlusion harder portion.

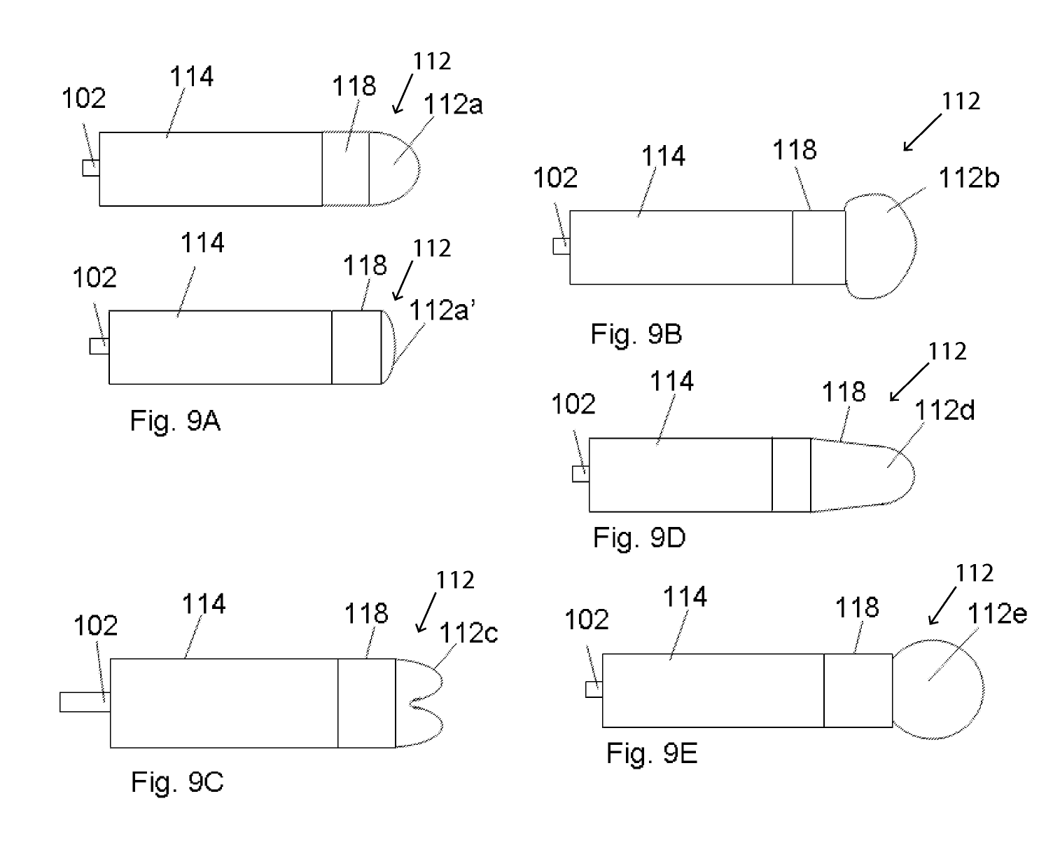

In some embodiments, the electrode tip 112 may be attached to the elongate member distal end. In other embodiments, laser welding is used to form electrode tip 112. In still other embodiments, other methods may be used to provide the electrode tip 112. In some embodiments the electrode tip 112 defines the electrode. In other words, the electrode tip 112 forms the energy delivery component of electrosurgical device 100. In one embodiment, the electrode tip 112 comprises a segment of a sphere, i.e. it is substantially spherical in shape. For example, the electrode tip 112 is a hemispherical or a rounded electrode tip 112 as shown in FIGS. 1-4, 8, 9A. In other words, the electrode tip 112 comprises a dome shaped electrode tip 112 which provides increased current density at the distal most tip, which facilitates higher arcing and allows the electrosurgical device 100 to cut through a relatively hard occlusion. The electrode tip 112 may comprise substantially of a segment of a sphere. In one example, the entire energy delivery portion comprising the electrode tip 112 (i.e. the entire surface from which energy is delivered) forms a segment of a sphere. As shown in FIG. 9A, electrode tip 112 comprises electrode tips 112a and 112a′ which each comprising substantially of a segment of a sphere.

In other embodiments, the electrode tip 112 may comprise a mushroom shaped tip 112b as shown in FIG. 9B. Furthermore, in some embodiments, the electrode tip 112 is substantially atraumatic, for e.g. as shown by electrode tips 112a and 112a′. In another embodiment, the electrode tip 112 may comprise a bi-arcuate electrode tip 112c, as shown in FIG. 9C. A laser weld process may be used that allows a cavity or bowl to be formed at the center of the bi-arcuate electrode tip 112c which may allow electrical energy and thus increased current density to be concentrated within the cavity. Thus arcing may be concentrated at the central region of the bi-arcuate electrode tip 112c. In one such embodiments, a support structure may be positioned adjacent the elongate member 102 distal end, distal to the heat shield 118 and laser welding may be used to form electrode tip 112c on it. Similarly, in some of the embodiments shown in FIGS. 9A-9D, the electrode tip 112 may be formed by laser-welding the distal end of the core wire 202 onto a support structure positioned distal to the heat shield 118. In a further embodiment, the electrode tip 112 forms a conical electrode tip 112d having a conical shape that tapers towards its distal end, as shown in FIG. 9D. This may allow arcing to be concentrated at the distal most end of the conical electrode tip 112d, which has a lower surface area and thus a higher current density. Additionally, in some such examples, as shown in FIG. 9D, the distal most end of the conical electrode tip 112d may be rounded in shape. This may allow the electrode tip 112 to be substantially atraumatic when inserted, for example, into the body vasculature. In an alternate embodiment, an electrode tip 112 may comprise a ball shaped electrode tip 112e, as shown in FIG. 9E. In some embodiments, the electrode tip 112 may have a surface geometry that allows sufficient current density to accumulate the electrode tip 112 that is sufficient to generate arcing to enable the electrosurgical device 100 to traverse through an occlusion. In some examples, the electrode tip 112 may be formed integrally with the elongate member 102. In other examples the electrode tip 112 may be otherwise attached to the elongate member 102 to form a secure connection therewith.

In some embodiments the electrode tip 112 is positioned distal to the heat shield 118. In some embodiments, the electrode tip 112 may be positioned adjacent the heat shield 118. In one example, the electrode tip 112 may be positioned distal to and adjacent to the distal face of the heat shield 118 as shown in FIGS. 9A-9E. In another embodiment, the electrode tip 112 is positioned distal to and adjacent a support structure 120 which may comprise an annular tubular structure through which core wire 202 is threaded as shown in FIGS. 1A, 1B and 2-4. In some embodiments, the electrode tip 112 may have an outer diameter that is equal or greater than the diameter of the distal components which are adjacent the electrode tip 112, such as the heat shield 1180R a combination of the heat shield 118 and the support structure 120. In other embodiments, the electrode tip 112 and additionally the support structure 120 both have an outer diameter which is equal to or greater than diameter of the heat shield 118. In such embodiments, when the electrode tip 112 is used to create a channel portion through tissue for e.g. an occlusion, by delivering energy through the electrode tip 112, a channel portion is created that is at least as wide as the electrode tip 112 outer diameter. This may allow at least the electrosurgical device distal region 104 to be advanced through the occlusion. In still other embodiments, the electrode tip 112 may have a diameter that is less than that of the heat shield 116 and/or the support structure 120. In some embodiments the electrode tip 112 helps retain the heat shield 118 in position within the device 100. In other words, the electrode tip 112 helps secure the heat shield 118 in place.

In some embodiments, as mentioned above, a support structure 120 is provided distal to the heat shield 118. The support structure 120 provides a distal surface on which the electrode tip 112 is positioned and/or formed. In one such embodiment, in order to create a dome shaped electrode tip 112 a welding process is used to melt a distal most portion of core wire 202 to form a segment of sphere for e.g. a hemispherical shape. In one specific example, a laser welding process is used and the support structure 120 provides a substantially planar distal face onto which the domed shaped electrode tip 112 is formed. In one instance of this example, the support structure 120 can withstand the laser welding process and can bond well with the core wire 202. The laser welding process allows the electrode tip 112 to fuse with the support structure 120 at the interface between the two. Additionally, the support structure 120 may have sufficient mechanical strength allowing it to be machined. Both the core wire 202 and the support structure 120 may be formed from biocompatible materials. In one specific example, the support structure 120 comprises a metal such as Tantalum and the core wire 202 comprises Nitinol. When the Nitinol core wire 202 is laser welded it fuses with the Tantalum support structure 120 at the interface between the two materials. An integral bond is formed at the boundary between the Nitinol electrode tip 112 and the tantalum support structure 120. The Tantalum support structure 120 allows the dome of the Nitinol electrode tip 112 to be formed on a flat surface. In other words, the Tantalum support structure 120 functions as a base to allow the Nitinol electrode tip 112 to be formed onto it. In one example, the electrode tip 112 may have an outer diameter (OD) of between about 0.027″ to about 0.032″ and a longitudinal length of between about 0.15 mm to about 0.20 mm.

In some embodiments, the support structure 120 may comprise materials such as tantalum, iridium, gold or stainless steel. In other embodiments, any other suitable material may be used. In still other embodiments, the support structure 120 may comprise any suitable material that is biocompatible and which may additionally have mechanical strength. In one example, the support structure 120 is radiopaque and provides the physician with a visual indication of the location of the electrode tip 112 under imaging. This helps determine the location of electrode tip 112 within the patient's body during use. In one specific example, an annular tubular structure comprising radiopaque tantalum metal is used as the support structure 120. The support structure 120 is threaded onto the distal end of the core wire 202 and the electrode tip 112 is positioned or formed distal to and adjacent to the support structure 120, the support structure 120 being positioned distal to heat shield 118. The tantalum support structure 120 has an inner diameter of about 0.279 mm, an outer diameter of about 0.812 mm, and has a longitudinal length of about 0.254 mm.

In one embodiment, the support structure 120 is electrically conductive and forms an electrode. Thus, the support structure 120 together with the electrode tip 112 forms the energy delivery component. In one example, a Nitinol electrode tip 112 is formed on a tantalum support structure 120 that is positioned distal to the heat shield 118. The electrode tip 112 is shaped to provide current density sufficient to generate arcing to create a channel through a region of tissue. Additionally, there is arcing generated at the tantalum support structure 120 on its sides and at the edges/corners of the support structure 120 at a discontinuity. In some examples, the heat shield 118 is flush with the support structure 120. In one example, the heat shield may not be positioned such that it is flush with the support structure 120 and there may be arcing at the junction/boundary between support structure 120 and the heat shield 118. In other words, if there is a gap at the proximal boundary of the support structure 120, there may be some arcing generated there. In one example, a ceramic filler may be used at this junction to fill the gap. In one specific example, if there is coagulum formation at the electrode tip 112 then arcing may be generated at the support structure 120. In another example, a substantially thin support structure 120 is used and arcing may be generated at the support structure 120. In some embodiments, the arcing observed at the support structure 120 in addition to arcing at the electrode tip 112, may help device 100 to traverse through a relatively large occlusion. In some embodiments the support structure 120 may not be electrically conductive. In some embodiments, the support structure 120 may be flush with and positioned adjacent the electrode tip 112. In some embodiments the support structure 120 and the electrode tip may be attached or secured. In other embodiments, the support structure 120 and the electrode tip 112 may not be attached. In one example, the support structure 120 is electrically conductive and forms an electrode together with electrode tip 112. In another example, the electrode tip 112 substantially forms the electrode. Thus, the electrode tip 1120R the support structure 120 in conjunction with the electrode tip 112, define the energy delivery component of electrosurgical device 100 through which energy can be delivered, for e.g. to a region of tissue within a patient's body.

An electrode tip 112 that is shaped substantially like a segment of a sphere, for e.g. a hemisphere, provides a sufficiently small surface area at the tip which may help support a sufficiently high current density allowing for effective arcing at the electrode tip 112. The shape and surface area of the electrode tip 112, allow the current density, and thus arcing to be focused/concentrated at the distal most tip. Thus, the electrode tip 112 may be configured and sized to have a surface geometry that allows sufficient current density to accumulate the electrode tip 112 that is sufficient to generate arcing to enable the electrosurgical device 100 to traverse through an occlusion. In other words the electrode tip 112 is configured and sized to such that electrical arcing is generated during the delivery of energy to create a channel through at least a portion of a region of tissue. In one embodiment an electrode may be formed integrally by the electrode tip 112 and the support structure 120. As mentioned previously, the support structure 120 may be electrically conductive and together with the electrode tip 112 may form an electrode. In some embodiments, the electrode tip 112 may have any of the shapes shown in FIGS. 9A-9D and may be positioned adjacent of the distal surface of the support structure 120 or the heat shield 118, as shown.

Various methods may be used to provide an electrode tip 112 that is shaped substantially like a segment of sphere. This may include an electrode tip 112 that is rounded or hemispherical in shape. In some embodiments, the electrode tip 112 that is shaped like a segment of a sphere may be removably attached to the distal end of the electrosurgical device 100. In one example, the electrode tip 112 may be a rounded cap electrode and may be removably affixed to the core wire 202 and/or to the support structure 120. Alternatively, in some embodiments, an electrode tip 112 may be coupled/attached directly to a distal end of the elongate member 102 distal to and adjacent to the heat shield 118. As mentioned above, in other embodiments the electrode tip 112 may have surface geometries similar to those shown in FIGS. 9A-9D. In one embodiment, a friction fit may be used such that the electrode tip 112 co-operatively engages with either the elongate member 102 and/or the heat shield 118. In one embodiment, the electrode tip 112 may be mechanically secured to the elongate member 102 and may be abutted against the heat shield 118 such that it rests against the heat shield 118. In one example, the heat shield 118 extends substantially radially along a proximal face of the electrode tip 112. In one example, a platinum band may be positioned proximal to the heat shield 118. In one example, the elongate member 102 may comprise a Nitinol wire and the electrode tip 112 may comprise a rounded Nitinol cap and may be attached to the distal tip of core wire 202. In one example, the electrode tip 112 comprises a Nitinol ball. A hole may be created/grinded in the ball and the core wire 202 may be received within the hole and attached thereto. In one embodiment, the electrode tip 112 may be may be secured to the elongate member 102 and/or heat shield 118 by an adhesive. In one example, the adhesive may comprise an epoxy. In one embodiment, the electrode tip 112 may be attached to core wire 202 using a melt-processing method. In some embodiments, the electrode tip 112 may be configured and sized to have a surface geometry that allows sufficient current density to accumulate at the tip that is sufficient to generate arcing to enable the electrosurgical device 100 to traverse through an occlusion. Thus in some embodiments, the heat shield 118 may be positioned proximal to the electrode tip 112, or proximal to the support structure 120 that supports the electrode tip 112. In one example, the heat shield 118 extends substantially radially along a proximal face of the electrode tip 112. In one example, the heat shield 118 extends substantially radially along a proximal face of an annular structure such as support structure 120. In one specific example, the support structure 120 is an annular structure which is electrically conductive and forms a part of the electrode.

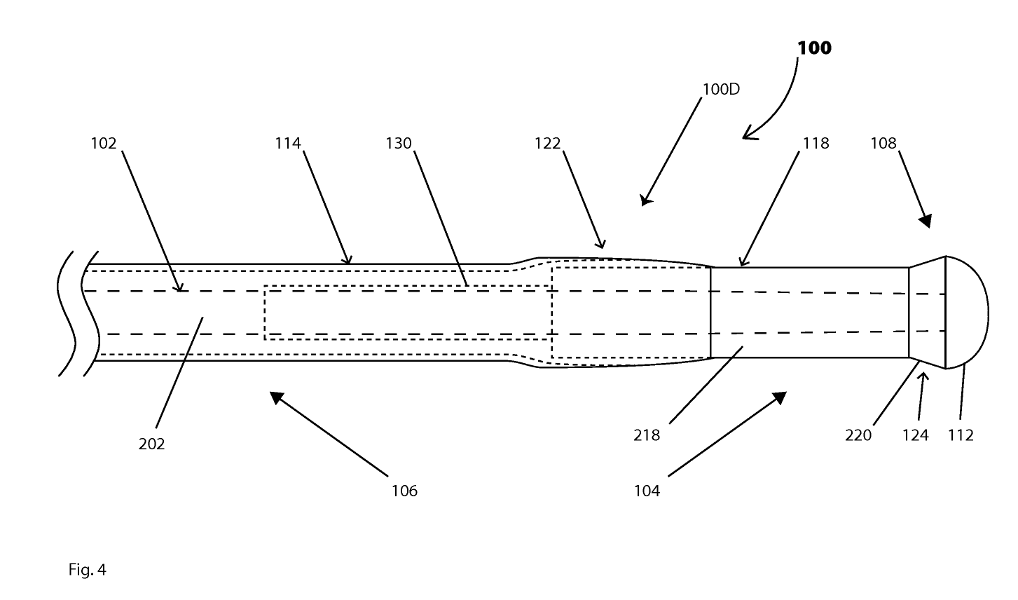

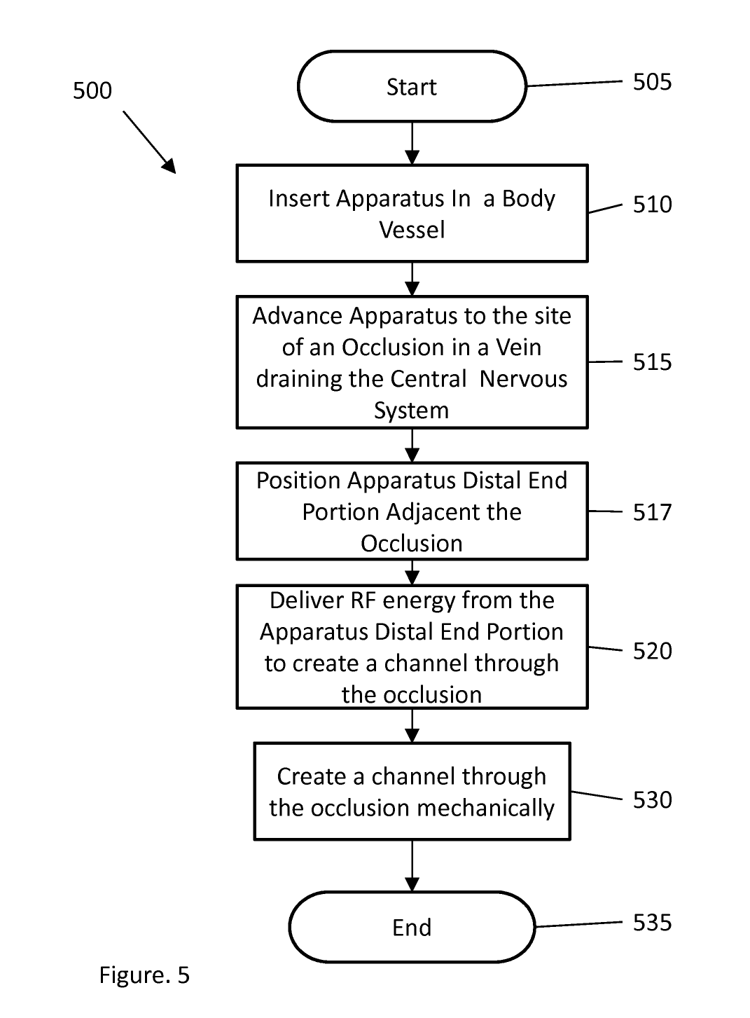

In an alternative embodiment of the present invention, as shown in FIG. 5, a support structure 220 is provided that comprises a tapered profile 124. The support structure 220 may have the properties as discussed above for support structure 120. In one example, the support structure 220 comprises an annular structure that has an outer diameter that gradually tapers from its distal end towards its proximal end as shown in FIG. 4. In one embodiment, the tapered support structure 220 has a distal outer diameter (OD) that matches the outer diameter of the distal electrode tip 112. Furthermore, the tapered support structure 220 has a proximal outer diameter (OD) that matches the outer diameter of the heat shield 118. Thus, a smooth outer profile is created by the tapered support structure 220 which facilitates the advancement of device 100D within tissue within a patient's body. The tapered profile 124 minimizes the risk of tissue snagging at the proximal boundary of the support structure 220. This minimizes the risk of charred tissue getting caught proximal to the electrode tip 112 and the support structure 220. This may further facilitate forward advancement of the device 100D as tissue is targeted by the active electrode tip 112 at the distal tip 108. The tapered profile 124 may also facilitate backward movement or retraction of device 100D during use, for example, within a patient's body (for e.g. within a vessel lumen). In one example, the tapered support structure 220 comprises tantalum. Thus, the tapered profile of tantalum having a distal OD larger than its proximal OD may allow easier traversal through the occlusion. The distal OD of tantalum may be greater than the device 100D OD along a portion of the device 100D proximal to the support structure.

In one embodiment of the present invention, the electrosurgical device 100, as shown in FIGS. 1A-4, may have an outer diameter (OD) at the distal tip 108 which is greater than the outer diameter (OD) along a portion of the device 100 proximal to the distal tip 108, such as OD of heat shield 118. The wider OD at the distal tip 108 may help facilitate traversal of the electrosurgical device 100 through an occlusion. In one example, during use, the larger distal tip OD of device 100 can create a puncture in tissue that is greater than the device OD proximal to the distal tip 108. In other words, the larger distal tip OD allows a channel to be created through tissue, for example an occlusion that is wider than a segment of device 100 proximal to the distal tip 108. Thus, the wider OD at the distal tip allows the device 100 to traverse easily through the occlusion with minimal risk of hindrance in the device path. This may allow the device 100 to cross with ease through the channel created. The larger distal OD also minimizes the risk of tissue being caught at the junction 122, for e.g. at the over-lap of the insulation layer 114 with the heat shield 118. Furthermore, a portion of the device proximal region 106 may have an outer diameter (OD) that is greater than the distal tip OD. This may allow the device 100 to further dilate the occlusion. In one example, the proximal portion of the device proximal region 106 has an OD that is wider than the distal tip OD. Additionally, in one example, the device proximal region 106 has a tapering profile that increases in diameter towards its proximal portion.

In accordance with an embodiment of the present invention, electrosurgical device 100 allows traversal through occlusions, which may include occlusion harder portions and occlusion softer portions. The electrosurgical device 100 provides a dome-shaped electrode tip 112 which provides sufficiently intense arcing to facilitate crossing of the device 100 through the harder part of the occlusion. In other words the rounded electrode tip 112 provides a sufficiently decreased surface area to facilitate arcing that allows the electrosurgical device 100 to traverse at least partially through an occlusion. As mentioned previously, the dome shaped electrode tip 112 may be formed on a support structure 120 which may be electrically conductive. The support structure 120 is mounted adjacent the heat shield 118 which is positioned distal to the electrical insulation layer 114. The heat shield 118 positioned distal to the electrical insulation may prevent the insulation layer 114 from degrading due to arcing observed at the electrode tip 112, as the device 100 is used to create a channel through an occlusion, for e.g. an occlusion harder portion.

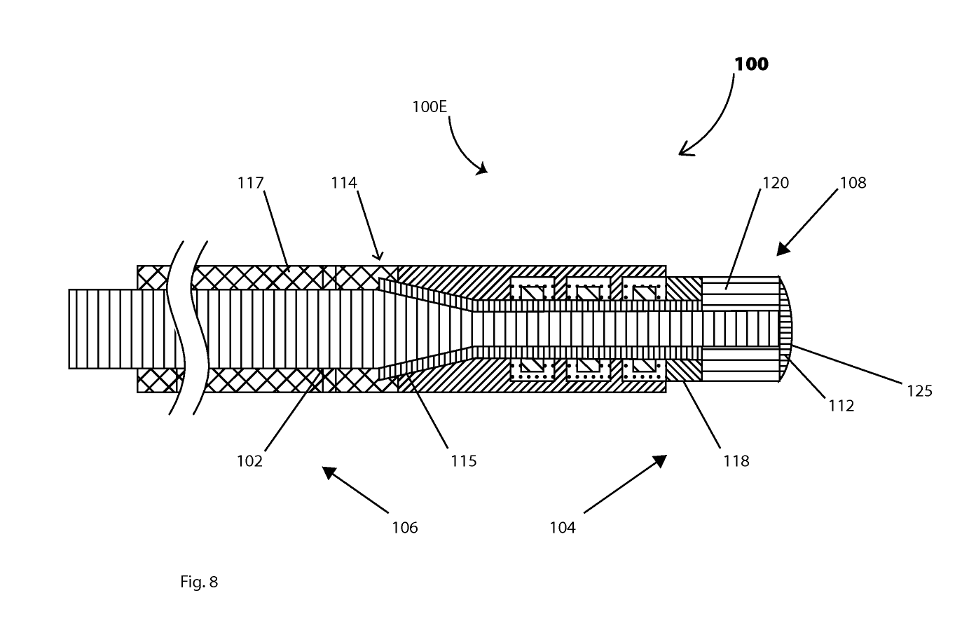

Referring to FIG. 8, the energy delivery apparatus, such as electrosurgical device 100E includes an elongate member or an electrical conductor 102, such as a substantially elongated electrical conductor, which may be any suitable conductor, such as a wire or a cable made out of a suitable electrically conducting material, such as for example, Nitinol, stainless steel, gold, platinum, titanium, silver or alloys thereof. The electrical conductor or elongate member 102 is substantially elongated and defines a conductor proximal end and a substantially longitudinally opposed conductor distal end. An electrode tip 112 is electrically coupled to the electrical conductor or elongate member 102 and located at a predetermined location therealong, for example adjacent to conductor distal end. The electrode tip 112 is provided for delivering electrical energy at a target location.

A proximal region 106 of the electrosurgical device such as a proximal region of the electrical conductor or elongate member 102 having an electrically insulating layer disposed thereon, is positioned in a substantially spaced apart relationship relative to the electrode tip 112.

Spacing apart the proximal region 106 of the electrosurgical device 100 from the electrode tip 112 ensures that any temperature increase caused by the delivery of electrical energy to the target location only minimally affects the device proximal region 106.

In the embodiment of the invention shown in FIG. 8, the proximal region 106 of the electrosurgical device is substantially longitudinally spaced apart from the electrode tip 112. More specifically, the electrode tip 112 is located distally relatively to the device proximal region 106. For example, the electrode tip 112 is located substantially adjacent to the conductor distal end. It is within the scope of the invention to have an electrode tip 112 that is formed integrally by a section of the outermost surface of the electrical conductor or elongate member 102.

In this embodiment, the electrode tip 112 defines tip distal surface 125 that is shaped substantially similarly to a portion of a sphere, i.e. rounded. This helps to ensure that injuries that may be caused to the body vessels, through movements of the electrode tip 112 through these vessels, are minimized.

In some embodiments of the invention, the energy delivery apparatus 100E includes an electrically insulating material substantially covering the electrical conductor or elongate member 102, such as for example and non-limitingly, Teflons®, such as polytetrafluoroethylene (PTFE), fluorinated ethylene propylene copolymer (FEP), perfluoroalkoxy (PFA), or ethylene and tetrafluoroethylene copolymer (ETFE, for example Tefzel®), or coatings other than Teflons®, such as polyetheretherketone plastics (PEEK™), parylene, certain ceramics, or polyethylene terpthalate (PET). In some embodiments, the electrically insulating material forms a layer that extends substantially radially outwardly from the electrical conductor 102. The electrically insulating material is described in further details hereinbelow.

In some embodiments of the invention, the energy delivery apparatus 100E further includes a heat shield 118 made out of a substantially thermally insulating material, for example, and non-limitingly, polytetrafluoroethylene (PTFE), which has a thermal conductivity of about 0.3 W/m-K. In this embodiment, the heat shield 118 may have a thickness of at least about 0.025 mm. In other embodiments, the thickness of the heat shield 118 may vary, depending on the thermal conductivity of the material being used. The heat shield 118 is located, at least in part, between the electrode tip 112 and the device proximal region 106. The heat shield 118 is provided for further thermally insulating the device proximal region 106 (having an elongate member 102 with an electrically insulating layer disposed thereon) from the electrode tip 112 and from heat produced by the delivery of electrical energy through the electrode tip 112.

In some embodiments of the invention, the heat shield includes polytetrafluoroethylene (PTFE). The use of PTFE is advantageous as, in addition to having suitable thermal insulation properties, PTFE is also an electrically insulating material (having a dielectric strength of about 24 kV/mm) and, therefore, contributes to the prevention of arcing between the electrode and any metallic material that may be present in the device proximal region 106. In alternate embodiments, other materials, such as for example, Zirconium Oxide, may be used for heat shield 118.

In one embodiment of the invention, the heat shield 118 extends substantially longitudinally from and contacts both the device proximal region 106 and the electrode tip 112. In other words, the heat shield 118 substantially fills a gap between the electrode tip 112 and the device proximal region 106. However, in alternative embodiments of the invention, the heat shield 118 extends substantially longitudinally only from one of the device proximal region 106 and the electrode tip 112 or, alternatively, the heat shield 118 does not contact either one of the device proximal region 106 and the electrode tip 112.

As shown in the drawings, the heat shield 118 is substantially annular and extends substantially radially outwardly away from the electrically insulating material covering the electrical conductor 102. In a very specific embodiment of the invention, the heat shield 118 is substantially annular and has a substantially similar outer diameter as device proximal region 106 and the electrode tip 112. This configuration results in an energy delivery apparatus 100E for which a distal region thereof has a substantially uniform outer diameter, which therefore facilitates navigation of the energy delivery apparatus 100E through body vessels and the creation of channels through occlusions and other biological tissues inside the patient. However, in alternative embodiments of the invention, the heat shield 118, the electrode tip 112 and the device proximal region 106 may all have any other suitable diameters.

In alternative embodiments of the invention, the electrical conductor 102 is made more flexible substantially adjacent the conductor distal end than substantially adjacent the conductor proximal end in any other suitable manner such as, for example, by using different materials for manufacturing the conductor proximal and distal regions. It has been found that a suitable material for manufacturing the electrical conductor 102 is Nitinol. Indeed, Nitinol shows super-elastic properties and is therefore particularly suitable for applying relatively large deformations thereto in order to guide the energy delivery apparatus 100E through relatively tortuous paths. Also, since the energy delivery apparatus 100E typically creates channels inside biological tissues through radio frequency perforations, in some embodiments of the invention, the energy delivery apparatus 100E typically does not need to be very rigid.

In some embodiments of the invention, the electrical insulation layer or electrically insulating material 114 is divided into a first electrically insulating material and a second electrically insulating material. A first electrically insulating layer or an outer polymer layer 117 made out of the first electrically insulating material substantially covers a first section of the electrical conductor 102. A second electrically insulating layer or an inner polymer layer 115 made out of the second electrically insulating material substantially covers a second section of the electrical conductor 102. The second section is located distally relatively to the first section. Furthermore, the first and second electrically insulating materials may comprise different materials with differing physical properties. For example, in some embodiments, the second electrically insulating material comprises polyimide, while the first electrically insulating material comprises PTFE. This allows for the second electrically insulating layer or inner polymer layer 115 to be substantially thinner than the first electrically insulating layer or outer polymer layer 117, while being sufficiently insulative so as to prevent undesired leakage of current. This substantially increases the flexibility of the energy delivery apparatus or electrosurgical device 100 substantially adjacent the apparatus distal end portion such as distal tip 108. In addition, this provides a material that is substantially more lubricious over the wider section of the energy delivery apparatus 100E so as to facilitate movement of the energy delivery apparatus 100E through body vessels and through channels created within the body.

### Applications

An embodiment of a treatment method of the present invention may be useful, for example, to penetrate through a material at least partly occluding a vessel of a body of a patient in order to recannalize the vessel. In a specific example the vessel comprises a vein draining the central nervous system. In such an example, the material to be penetrated may comprise a vascular occlusion having regions of various degrees of toughness and calcification. In accordance with the embodiments described herein below, an occlusion may refer to chronic total occlusions, partial occlusions, blockages or conditions that cause narrowing of the blood vessel such as vascular malformations or stenosis. Thus, the term “occlusion” may refer to any obstruction that at least partially restricts blood flow through the vein.

As mentioned above, in some examples a venous occlusion may be present in the veins that drain the central nervous system compromising the flow of blood within these veins leading to chronic cerebrospinal venous insufficiency (CCSVI). In a particular example of this, the azygos and/or the internal jugular veins may be occluded or stenosed (abnormally narrowed). In other examples, the occlusion may involve the subclavian and/or brachiocephalic/innominate veins. Furthermore, truncular venous malformations, including azygous stenosis, defective jugular valves and jugular vein aneurysms, and conditions within the superior vena cava such as stenosis may also to contribute to CCSVI. The internal jugular veins (IJV), subclavian veins (SCV), brachiocephalic veins (BCV) and the superior vena cava (SVC) are all illustrated in FIGS. 6A-6C.

Embodiments of the present invention provide an apparatus such as device 100 as described herein above to treat an occlusion within a body vessel. Referring now to FIGS. 5 and 6A-6C, in a more specific example of implementation, the present invention is embodied in a method 500 for creating a channel 600 through an occlusion 602 located in a substantially elongated body vessel 604 of a patient, the vessel comprising a vein that drains the central nervous system such as the internal jugular vein (IJV), the occlusion 602 extending substantially longitudinally relative to the body vessel 604. For example, as shown in FIGS. 6A and 6B, the occlusion 602 may be in the left internal jugular vein of the patient (IJV), and may additionally extend into other veins as shown in FIGS. 6B and 6C. FIG. 6C illustrates an occlusion 602 within the left IJV that curls into the left subclavian vein towards the brachiocephalic vein.

It has been found that embodiments of the proposed method are well suited, for example, to the creation of channels through plaque partially or totally occluding veins draining the central nervous system such as the IJV. Such occlusions are often tough, calcified and/or difficult to traverse using a mechanical guide wire. The person skilled in the art will readily appreciate that the channel 600 is not necessarily a self-supporting channel 600. Therefore, in some embodiments of the invention, the channel 600 is further dilated or receives a stent, or is both dilated and receives a stent, after having been created. In still other embodiments, the channel 600 may be created through an occlusion located within a region of the IJV previously having a stent or other conduit placed therein, for example after treatment of a previous occlusion or an aneurism.

The method 500 uses a channel creating apparatus, such as, for example, an embodiment of the apparatus described hereinabove such as electrosurgical device 100, defining an apparatus distal end portion, such as for example the distal tip 108 described hereinabove, insertable into the body vessel. The channel creating apparatus, such as electrosurgical device 100 includes an energy delivery component, such as electrode tip 112 operatively coupled to the apparatus distal end portion for delivering energy substantially adjacent the apparatus distal end portion.

The method 500 starts at step 505. Then, at step 510, the apparatus distal end portion is inserted into a body vessel. Afterwards, at step 515, the apparatus is advanced to the site of the occlusion in a body vessel 604 comprising a vein that drains the central nervous system, such as the IJV, as shown in FIG. 6A and discussed in further detail herein below with respect to an exemplary embodiment of a method of the present invention. At step 517, the distal end portion is oriented to follow a trajectory of the vein to steer the apparatus distal end portion and the apparatus is positioned such that apparatus distal end portion is located substantially adjacent the occlusion 602 within the body vessel 604 for example the IJV. At step 520, the apparatus or device 100 is used to deliver energy, which in one example may be radiofrequency (RF) energy. The radiofrequency energy is delivered for example through the electrode tip 112 at the apparatus distal end portion to create a channel 600 through the occlusion.

The creation of the channel 600 through delivery of energy may restore blood flow or improve blood flow within the IJV. The energy delivered by the apparatus distal end portion may not be limited to radiofrequency (RF) energy and may comprise other forms of energy. For example, during the delivery of RF energy the apparatus distal end portion may be pushed by applying a substantially longitudinal force to an apparatus proximal portion (for example, the proximal region of the apparatus described hereinabove) longitudinally opposed to the apparatus distal end portion. In another example, the apparatus distal end portion is pushed directly, for example using a motor or any other actuator coupled to the apparatus distal end portion. It should be noted that while embodiments of the apparatus described hereinabove are usable to perform the method 500, in other embodiments of the invention, any suitable apparatus may be used. Subsequently, at step 530, in some embodiments the channel 600 through the occlusion 602 may then be created or extended mechanically. In one example, the apparatus distal end portion is positioned substantially adjacent the occlusion and the channel may be created mechanically by pushing the apparatus distal end portion through at least a portion of the occlusion 602, the channel 600 being created substantially without using the energy delivery component such as electrode tip 112, to deliver energy into the occlusion 602.

Also, while in the method 500 the channel 600 is initially created using the delivery of RF and subsequently the channel 600 is extended mechanically, it is within the scope of the invention to create the channel mechanically intitially before the channel 600 is extended using RF energy. In other words in some embodiments, there may be a step of attempting to cross the occlusion 602 mechanically prior to a step of creating a channel through the occlusion 602 using delivery of RF. It has been found that the method embodied in FIGS. 5 and 6A-6C leads to a new and unexpected result in which the channel 600 is created through a vein that drains the central nervous system relatively easily, safely and effectively using an apparatus that is capable of delivering RF energy, such as electrosurgical device 100. Furthermore, electrosurgical device 100 may additionally have mechanical properties that are substantially similar to those of standard mechanical guide-wires. Other advantages of the proposed methods have been mentioned hereinabove. Thus the device and method of the present invention may be particularly useful, for example, where the occlusion within the veins draining the central nervous system is tough or difficult to traverse using a mechanical guide.

For example, it was found generally that when an occlusion 602 has a hardness such that a mechanical pressure of at least about 20 kg/cm2 is required to create a channel thereinto, conventional mechanical guide-wires are typically unusable to create the channel 600. The embodiments of the present invention thus provide, amongst other advantages, a means of delivering RF energy for traversing an occlusion within veins that drain the central nervous tissue that standard mechanical guide-wires may be unable to penetrate.

In some embodiments of the invention, step 520 is performed as follows. First, the channel 600 is created through the occlusion 602 while delivering the energy into the occlusion 602 and the apparatus distal end portion is advanced through the channel 600. The advancement of the apparatus distal end portion may be substantially simultaneous/concurrent with or subsequent to the delivery of energy. When a portion of the channel 600 has been created, the delivery of energy is stopped. Afterwards, a user of the channel creating apparatus may attempt to traverse the occlusion mechanically as discussed herein above with respect to step 530. In one example, the user may push the apparatus distal end portion through the occlusion 602 substantially without delivering energy via the energy delivery component such as electrode tip 112. In another example, at step 530 the user may attempt to cross the occlusion 602 mechanically by withdrawing the apparatus from the body vessel 604 and advancing a mechanical guide wire to the site of the occlusion. The user may then attempt to cross the occlusion 602 by pushing through the occlusion using the mechanical guide wire. Upon the apparatus distal end portion being unable to be pushed through the occlusion 602 mechanically, or if the mechanical guide wire is unable to cross the occlusion 602 the channel 600 may be created/extended through the occlusion 602 using RF delivered through apparatus distal end portion after the apparatus such as device 100 has been re-advanced to the site of the occlusion, similar to the initial creation of channel 600 using RF energy as described herein above. If required, additional segments of the channel 600 are also similarly created. Thus, in some embodiments steps 520 to 530 may be repeated until at least a portion of the occlusion has been traversed. In some embodiments the steps 520 to 530 may be repeated until the entire occlusion 602 has been traversed. The procedure may then be stopped at step 535.

Thus, as described hereinabove, in some embodiments, the user attempts to cross the occlusion mechanically prior to and/or after the step of creating a channel by delivering energy through the apparatus distal end portion. In one such example, the user may advance a mechanical guide wire to the site of the occlusion to attempt to cross the occlusion mechanically. Alternatively, in another example, the user may advance apparatus such as device 100 to the site of the occlusion and may attempt to cross the occlusion mechanically using a channel creating apparatus or device 100 by pushing the apparatus distal end portion mechanically through at least a portion of the occlusion 602 substantially without the delivery of RF energy.

Therefore, in some embodiments, by repeatedly testing for the possibility of creating the channel mechanically after, for example, each energy delivery step, an intended user may lower the risk that the energy delivered may injure tissues that should remain intact, such as for example a vessel wall of the body vessel 604 such as a vein that drains the central nervous system such as the IJV.

In some embodiments of the invention, the energy is delivered for a predetermined amount of time prior to stopping the delivery of the energy. In other embodiments, the user may decide, during the course of the procedure, on the amount of time during which energy should be delivered. For example, the amount of time during which energy may be delivered may be from about 0.1 seconds to about 5 seconds. In a more specific embodiment of the invention, the amount of time during which energy is delivered for a duration of from about 0.5 seconds to about 3 seconds. More specifically, from between about 0.5 seconds to about 2.5 seconds. It has been found that these amounts of time allow for the creation of the channel 600 in a reasonable amount of time while reducing the risk of unwanted injuries. During the periods of time described above, the energy may be delivered continuously or as a pulsed waveform.

In one specific embodiment, once the apparatus such as device 100 has been advanced into the vein such as the IJV at the site of the occlusion, radiofrequency energy may be delivered from the device 100. The device may be connected to a radiofrequency generator that allows delivery of energy in a pulsed manner or delivers energy with a 2-second burst of RF which may allow a more controlled approach to traversing through the occlusion than using mechanical force and traditional guide wires.

In other embodiments of the invention, when performing step 520, the intended user assesses continuously, periodically or intermittently the position of the apparatus distal end portion relatively to the occlusion 602. For example, the position of the apparatus distal end portion is assessed using a position assessment method selected from the group consisting of an imaging technique, an impedance measurement, a measurement of a force exerted onto the apparatus distal end portion, a measurement of a pressure exerted onto the apparatus distal end portion and a measurement based on ultrasonic signals, among other possibilities. In these embodiments, assessing the position of the apparatus distal end portion may allow the user to deliver energy to the occlusion 602 over a minimal duration, which again may lower the risk of injuring structures adjacent to the occlusion 602.

In some embodiments, if a user is unable to advance the apparatus through the occlusion 602, the user may attempt to re-orient at least a portion of the apparatus. As described hereinabove, such re-orientation may take the form of steering the device in some manner or, alternatively, applying torque to a portion of the device. In some such embodiments, the position of the apparatus may be assessed, as described hereinabove, prior to choosing which course of action to follow.

As mentioned hereinabove, the device of the present invention is capable of delivering energy and the energy delivered may be any suitable energy. For example, the energy may be radio-frequency (RF) electromagnetic energy (as mention previously) or radiant energy, for example optical energy such as laser light, ultrasonic or vibrational energy, among other possibilities, to allow apparatus such as device 100 traverse through an occlusion within veins draining the central nervous system. For example, when radio-frequency energy is used, it has been found that radio-frequency energy delivered with a power of at least about 5 W at a voltage of at least about 75 Volts (peak-to-peak) produces good channel creation performances while remaining relatively safe for the patient.

In some embodiments of the invention, the energy delivery component, such as electrode tip 112 is selectively operable in an energy delivering state and a deactivated state. In the energy delivering state, the energy is delivered substantially adjacent the apparatus distal end portion. In the deactivated state, the energy is substantially not delivered substantially adjacent the apparatus distal end portion.

When such an energy delivery component, such as electrode tip 112 is used, the method 500 may be performed such that the channel 600 is initially created through the occlusion 602 by operating the energy delivery component, such as electrode tip 112 in the energy delivering state and delivering the energy into the occlusion 602. The channel 600 may be continued to be created through the occlusion 602 mechanically, either by inserting a mechanical guide wire or by operating the energy delivery component, such as electrode tip 112 in the deactivated state and pushing the apparatus distal end portion through at least a portion of the occlusion 602.

An advantageous, but non-limiting, embodiment of the invention using such an energy delivery component, such as electrode tip 112, is one wherein the channel creating apparatus includes a pressure sensor operatively coupled to the apparatus distal end portion for measuring a pressure exerted onto the occlusion by the apparatus distal end portion. Then, in some embodiments of the invention, the method 500 includes operating the energy delivery component, such as electrode tip 112 in the energy delivering state if the measured pressure is substantially above a predetermined pressure and operating the energy delivery component, such as electrode tip 112 in the deactivated state to cross the occlusion mechanically or utilizing a mechanical guide wire if the measured pressure is substantially below the predetermined pressure. The predetermined pressure is, for example, the pressure at which mechanical penetration is difficult or impossible. The energy delivery is manually switched on or off, or the channel creating apparatus includes a controller for automatically turning the energy delivering apparatus to the deactivated state if the pressure exerted onto the occlusion by the apparatus distal end portion is substantially below the predetermined pressure and turning the energy delivery apparatus to the energy delivering state if the pressure exerted onto the occlusion by the apparatus distal end portion is substantially above a predetermined pressure.

In these embodiments, the activation of energy delivery occurs only if the energy is required to create the channel 600 or a portion thereof. Otherwise, only a mechanical force is used to create the channel 600 either using a mechanical guide wire or apparatus such as device 100. This reduces uncertainty and variability in the manner in which the method is performed. Also, by reducing or eliminating the need to repeatedly test for the possibility of creating the channel mechanically, the method may be performed relatively fast with relatively low risks of injuring the patient.

In some embodiments of the invention, if an intended user is unable to push the mechanical guide wire or apparatus distal end portion mechanically through the occlusion 602, the intended user may re-orient the apparatus distal end portion within the channel 600 and attempt to push the apparatus distal end portion through the occlusion 602 substantially without delivering energy via the energy delivery component, such as electrode tip 112. In some embodiments of the invention, energy is used to facilitate the reorientation of the apparatus distal end portion.

In any or all of the embodiments described herein, a user may elect to initially use a standard mechanical guide-wire to attempt to penetrate an occlusion. Once a portion of the occlusion is encountered that is not amenable to being traversed mechanically (for example without the use of extensive or excessive force), the method 500 as described hereinabove may be employed.

In further detail, in some embodiments of the invention, the energy delivery apparatus such as electrosurgical device or energy delivery apparatus 100E is used such that a channel 600 is created at least partially through the occlusion. This channel may be created by delivering energy through the electrode tip 112 and advancing the apparatus distal end portion such as distal tip 108 into the occlusion 602 simultaneously or after delivering energy.

In some embodiments of the invention, when the intended user of the energy delivery apparatus such as electrosurgical device 100E finds that advancing through the occlusion 602 or any other material becomes relatively difficult, the intended user may retract the apparatus distal end portion and apply electrical energy while a gap exists between the apparatus distal end and the target location. Then, a channel 600 may be created more easily, for example due to the space created between the electrode tip 112 and the occlusion 602. Afterwards, the apparatus distal end portion may then be further advanced through this channel.

In some embodiments of the present invention, as mentioned previously energy may be delivered from the electrode tip 112 in either a continuous mode or a discontinuous mode. In one example, a continuous mode is used and power is supplied to sustain arcing for a period of about 3 seconds in order to facilitate traversal through an occlusion harder portion 606. In an alternate example, a discontinuous mode is used and power is supplied for a period of about 10 seconds. In one example, power is supplied at a frequency of 1 Hz.

If the occlusion 602 has been traversed during the initial delivery of energy, then the device may be advanced through the occlusion 602 with or without the use of energy or with a mechanical guide wire.

In one embodiment of the method of the present invention, the energy delivery device such as the electrosurgical device 100 is advanced to a target location such as within an elongated body vessel 604 within a patient's body such as a vein draining the central nervous system. The energy delivery portion such as electrode tip 112 is positioned adjacent an occlusion 602. As outlined above, energy is delivered through the energy delivery portion, such as electrode tip 112, to create arcing allowing device 100 to form a portion of the channel 600 through the occlusion 602. The electrode tip 112 and the electrosurgical device 100 may then be mechanically advanced or a mechanical guide wire may be used to traverse through the occlusion 602, to create another portion of channel 600.

In one embodiment, the electrosurgical device 100 is substantially rigid/stiff along a portion thereof. After a substantial segment of the occlusion 602 has been crossed, a dilation catheter may be advanced over the electrosurgical device 100, in order to further enhance/widen the channel 600. The electrosurgical device 100 is sufficiently stiff along a portion thereof to allow it to function as a rail allowing the dilation catheter to be advanced over it. The dilation catheter having a wider outer diameter along at least a portion thereof, allows the channel 600 within the occlusion 602 to be widened. The dilation catheter may then be withdrawn and a balloon catheter may then be advanced over the electrosurgical device and positioned within the channel 600 created by electrosurgical device 100. The balloon may then be inflated to further dilate or widen the channel 600. The dilation using a balloon catheter may be performed once or multiple times and may be repeated at various locations within the channel in order to dilate the channel 600. Furthermore, the dilation using the balloon catheter may be performed at multiples occlusion sites within the body vessel 604 and wherein the step of inflating the balloon is repeated at each of the occlusions.

In an alternate embodiment, the electrosurgical device 100 defines a device distal region 104 having has a distal end portion, such as distal tip 108, that has an outer diameter (OD) that is wider than the OD of a proximal portion of the device distal region 104. In one example, the distal end portion of the electrosurgical device has an outer diameter of 35 thousandths of an inch (“thou”) at the distal tip 108. The outer diameter of the device distal region 104 then tapers proximally to 25 thou. As mentioned previously, in one example, a proximal portion of the device proximal region 106 has an outer diameter that is wider than the distal tip 108 of the electrosurgical device 100. As the electrosurgical device 100 is advanced through the occlusion 602 the proximal portion of the device proximal region 106 functions to dilate the channel 600. This may help to minimize the step of inserting a dilating catheter over the electrosurgical device 100 in order to further dilate the occlusion. The electrosurgical device 100 thus functions both to traverse through the occlusion (both occlusion harder portions 606 and occlusion softer portions 608) by forming a channel 600 and additionally may function to dilate the channel 600 as it is formed. Thus the step of inserting a dilating catheter may be minimized. A balloon catheter may then be advanced directly over the electrosurgical device 100 and one or more inflations may be used to further wider the channel 600.

Furthermore, in some embodiments, the delivery of energy through the apparatus such as device 100 may be directed away from the vessel wall. In some embodiments the apparatus distal end portion is steerable to facilitate orienting the apparatus distal end portion. In other embodiments, the apparatus distal end portion is angled to facilitate the step of orienting the apparatus distal end portion. As mentioned herein above, the apparatus such as device 100 may be curved or bent at its distal end. This may allow the device to be maneuvered through the vasculature to the target location at the site of the occlusion within the vein that drains the central nervous system, such as within the internal jugular vein (IJV). In some specific embodiments the distal end portion of the device or apparatus may be bent or curved at an angle of about 10 degrees to about 50 degrees from a longitudinal axis of the apparatus. More specifically, the apparatus distal end portion has an angle of between about 20 degrees to about 40 degrees with respect to the longitudinal axis of the apparatus. In a specific example of this, the apparatus such as device 100 is kinked (i.e. has a sharp bend) with an angle of about 20 degrees from the longitudinal axis. In another example, the apparatus such as device 100 is kinked an angle of about 30 degrees from the longitudinal axis. In still an additional example, the apparatus has a gradual curve where the distal end portion is at an angle of about 40 degrees from the longitudinal axis. The angled distal end portion allows the device to be advanced to the target occlusion in tortuous anatomy. The curvature may allow more control over the trajectory of the apparatus as it is advanced while applying RF and also if a change in trajectory is required mid-occlusion. In one specific example, an apparatus distal end portion that is at an angle of about 40 degrees may allow access to tight brachiocephalic occlusions.

Furthermore, in embodiments where the apparatus such as device 100 is advanced into an occlusion within a region of a vein having a stent placed therein, a safety system may be provided to ensure that the RF energy is not activated when the apparatus distal end portion is too close to the stent. This may help avoid lesion formation by preventing RF energy from being transmitted through a low impedance metal object such as the stent. Thus, the delivery of energy from the energy delivery portion of the device such as the apparatus distal end portion may be prevented when the apparatus distal end portion is positioned adjacent to or in contact with a metal object such as a stent. In one specific example of this, the apparatus may be coupled to a generator that generates an error signal if the apparatus distal end portion touches the stent or is too close to the stent. In other words, if the energy delivery component of the apparatus is substantially contacting the stent, an error may be detected and the delivery of energy into the occlusion using the energy delivery component may be stopped. Further details of such a safety system may be found in U.S. utility patent application Ser. No. 13/410,868, filed on Mar. 2, 2012, which is incorporated herein by reference in its entirety. This allows the practitioner to guide the apparatus end portion into the occlusion away from the stent and towards the centre of the occlusion until the error signal is no longer observed.

### Example 1

In a specific example, the patient had a previous Left Internal Jugular Vein (LIJV) angioplasty and the physician was unsuccessful at re-canalizing the LIJV mechanically at a first attempt. A second attempt was successful at recanalizing the LIJV vein using an embodiment of a device 100 to deliver energy into the occlusion to create a channel therethrough. Additionally a stent was deployed within the LIJV at the site of the previous occlusion. Subsequently, following the procedure, the stented LIJV had later become re-occluded requiring use of device 100 to create a channel through the occlusion. The patient was diagnosed as having CCSVI and multiple sclerosis (MS) and presented with an occlusion of the LIJV approximately 3 cm in length, in addition to stenosis in the Right Internal Jugular Vein (RIJV) and azygous vein.

In some embodiments, a physician may use a micro-puncture kit to gain access to an appropriate vein that provides access to the internal jugular vein (IJV). In some embodiments access may be provided through another vessel in the body, for example through a puncture in a femoral or saphenous vein. In some embodiments the physician may use multiple points of access depending on the patient. In this specific example, access to the LIJV was provided using an introducer access sheath, specifically the 9F Pinnacle Access Sheath (Terumo), with a micro-puncture needle and wire, and access was gained through the patient's right groin. The patient had been placed under conscious sedation. After gaining access, pressures were measured in the renal and azygous veins and angioplasties performed if required before proceeding to investigate the internal jugularr veins.

The patient had an abnormally long neck with the subclavian vein (SCV) presenting at the plane of the sternum, while it should normally be much higher. After gaining access into the azygous vein from the superior vena cava (SVC) and then attempting to recanalize the occlusion from below, the physician proceeded to gain access in the RIJV. A 260 cm angled mechanical guidewire and 75 cm 4 F catheter were inserted and advanced into the RIJV. The patient's right side of the neck was acting as the primary drainage route for the head since the left side was blocked. The RIJV appeared relatively patent except for the base (J1 at 50%). The 4 F catheter was removed and a 14 mm by 4 cm long balloon was inserted and the right internal jugular vein was dilated. The balloon was subsequently removed.

The physician then positioned the guidewire in position on the left side within the LIJV and a contrast injection showed several collaterals around the stent and the stump of the total occlusion. An ultrasound of the neck showed that the occlusion within the LIJV was sonolucent which indicated that the occlusion may be a relatively recent occlusion. In some embodiments, for occlusions in the jugular veins the physician may insert a snare from the femoral access and advance a radiofrequency guidewire antegrade from a jugular access. The ultrasound of the neck served to assist the physician in attempting to gain an access point within the neck to approach the occlusion in such a top-down manner. However, a suitable vein was not found to access the LIJV as only the collaterals were visible. An attempt was then made to recanalize the occlusion within the LIJV from below with the guidewire being positioned within the LIJV.

An introducer/guide-catheter and catheter were then inserted over the guidewire. The guidewire was then swapped out for the RF guidewire 100 with a torquing device being loaded over the RF guidewire before it was plugged into the connector cable. More specifically, a 20° angled RF guidewire was connected to an electrical radiofrequency generator via a connector cable. Thus, the RF guidewire was guided to the site of the occlusion within the stent 700 within the left IJV from below as shown in FIG. 6A.

Initially, the occlusion appeared softer than the ultrasound suggested since the catheter traversed a portion of the occlusion mechanically, however it appeared that the occlusion became much more organized as it progressed in a cranial direction. At this point, the sheath was pointed toward the side of the stent but the angled catheter was pointed cranially. Five radiopaque bands of the RF guidewire were visible through the stent struts on imaging and the RF guidewire was then torqued to point away from the stent wall before it was retracted back into the catheter.

The generator was then turned ON and power was delivered at 25 Watts for 2 seconds. The Power window on the generator displayed fluctuations of 7, 5, and 1 Watt. This allowed the RF guidewire to advance a few centimeters somewhat medial from the center of the stent 700 (such that three of the radiopaque bands were now visible external to the catheter). A second delivery of energy was then applied at the same settings, resulting in displayed power fluctuations of 9, 3, 4 and 3 Watts. The RF guidewire was then advanced another few centimeters and the physician torqued the RF guide wire during advancement to direct it medially.

The RF guidewire is torquable and was torqued simultaneously while advancing to avoid extravascular perforations. The catheter was guided to the tip of the RF guidewire and energy delivery was then turned off.

A third application of power at the same settings resulted in observed power fluctuations of 2 and 7 Watts before resulting in an error code likely as a result of the RF guidewire being positioned adjacent to or in contact with the stent 700. As the RF guidewire was positioned too close to the stent, an error code was generated which resulted in the energy delivery from the generator being shut off.

A fourth application at the same settings resulted in an immediate error code. At this point, the physician concluded that the vasculature was getting too narrow to continue using RF as it would likely result in triggering additional error codes. This may be due to the stent struts being spaced much closer together at this specific section of the vessel. The physician then swapped the RF guidewire out and reinserted the mechanical guidewire. The mechanical guidewire was then used to attempt to continue traversing through the occlusion without the delivery of RF.

In this particular example, the occlusion within the left internal jugular vein was partially treated using both the radiofrequency guidewire as well as a mechanical guidewire in order to treat CCSVI.

Thus, in one broad aspect, embodiments of the present invention comprise a method for creating a channel through an occlusion located in a vein that drains the central nervous system, said method using a channel creating apparatus including an energy delivery component operatively coupled to a distal end portion of said channel creating apparatus, said method comprising: creating a channel through said occlusion by delivering energy into said occlusion using said energy delivery component to substantially increase blood flow through said vein to treat the chronic cerebrospinal venous insufficiency.

The embodiments of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

We claim:

1. A method for creating a channel through an occlusion located in a vein that drains the central nervous system of a patient, said method using a channel creating apparatus including an energy delivery component operatively coupled to a distal end portion of said channel creating apparatus, said method comprising:
creating a channel through said occlusion by delivering energy into said occlusion using said energy delivery component to substantially increase blood flow through said vein to treat chronic cerebrospinal venous insufficiency.

2. The method of claim 1, wherein said energy comprises radiofrequency electrical energy.

3. The method of claim 2, wherein said energy comprises pulsed radiofrequency energy.

4. The method of claim 2, wherein radiofrequency is delivered for between about 0.5 seconds to about 2.5 seconds.

5. The method of claim 1, wherein said vein is selected from the group consisting of: an internal jugular vein, an azygous vein, a subclavian vein and a brachiocephalic vein.

6. The method of claim 1, wherein said method further comprises, prior to creating the channel, a step of orienting the apparatus distal end portion within the vein to follow a trajectory of the vein to steer the apparatus distal end portion substantially adjacent said occlusion.

7. The method of claim 6, wherein said apparatus distal end portion is angled to facilitate the step of orienting the apparatus distal end portion.

8. The method of claim 7, wherein said apparatus distal end portion has an angle of between about 20 degrees to about 40 degrees with respect to a longitudinal axis of the apparatus.

9. The method of claim 1, the method further comprising a step of advancing a dilation catheter over the apparatus within the channel created by the apparatus in order to dilate the channel.

10. The method of claim 9, comprising the steps of:
withdrawing the dilation catheter;
advancing a balloon catheter over the apparatus within the channel created by the apparatus; and
inflating the balloon to further dilate the channel.

11. The method of claim 10, wherein the step of inflating the balloon is repeated at various locations within the channel in order to further dilate the channel.

12. The method of claim 10, wherein the vein includes multiple occlusions and wherein the step of inflating the balloon is repeated at each of said occlusions.

13. The method of claim 10, further comprising a step of deploying a stent within said channel.

14. The method of claim 1, further comprising a step of attempting to cross the occlusion mechanically prior to the step of creating a channel by delivering energy.

15. The method of claim 14, wherein the step of attempting to cross the occlusion mechanically is performed using a mechanical guide wire.

16. The method of claim 14, wherein the step of attempting to cross the occlusion mechanically is performed using the channel creating apparatus.

17. The method of claim 1, wherein said occlusion is located within a region of the vein having a stent.

18. The method of claim 17, further comprising a step of detecting an error and stopping the delivery of energy into said occlusion using said energy delivery component if the energy delivery component is substantially contacting the stent.

19. A method for creating a channel through a site of an occlusion located in a vein that drains the central nervous system of a patient, said occlusion at least partially restricting the flow of blood within the vein, said method using a channel creating apparatus comprising a radiofrequency guidewire, the channel creating apparatus including an energy delivery component operatively coupled to a distal end portion of said channel creating apparatus, said method comprising the steps of:
advancing a mechanical guide-wire within the vein substantially adjacent the occlusion;
applying contrast at the site of the occlusion to visualize the extent of the occlusion;
advancing the radiofrequency guidewire to the site of the occlusion such that the apparatus distal end portion is positioned adjacent the occlusion;
attempting to advance through the occlusion mechanically substantially without the delivery of radiofrequency energy using the radiofrequency guidewire; and
upon being unable to advance the radiofrequency guidewire mechanically, creating a channel through said occlusion by delivering energy into said occlusion using said energy delivery component to substantially increase blood flow through said vein.

20. The method of claim 19, wherein the steps of attempting to advancing through the occlusion mechanically and creating a channel through the occlusion are performed repeatedly in order to cross the occlusion to treat chronic cereprospinal venous insufficiency while lowering the risk that energy delivery may injure tissues that should remain intact.
